SEQUENCE LISTING (23 sequences)

SEQ ID NO: 1 - protein, 11 aa, Artificial sequence
  Synthetic
KIRLRPGGKKK

SEQ ID NO: 2 - protein, 50 aa, Artificial sequence
  Synthetic
YVWKTWGQYWQVLGGPVSGLSIGTGRAMLGTHTMEVTVYHRRGSRSYVPL

SEQ ID NO: 3 - protein, 50 aa, Artificial sequence
  Synthetic
SSGTLISRALVVTHTYLEPGPVTAQVVLQAAIPLTSCGSSPVPGTTDGHR

SEQ ID NO: 4 - protein, 51 aa, Artificial sequence
  Synthetic
DISHLLADVARFAEGLEKLKECVLHDDLLEARRPRAHECLGEALRVMHQII

SEQ ID NO: 5 - protein, 51 aa, Artificial sequence
  Synthetic
DISHLLADVARFAEGLEKLKECVLRDDLLEARRPRAHECLGEALRVMHQII

SEQ ID NO: 6 - protein, 50 aa, Artificial sequence
  Synthetic
ASVLSGGKLDRWEKIRLRPGGKKKYKLKHIVWASRELERFAVNPGLLETS

SEQ ID NO: 7 - protein, 50 aa, Artificial sequence
  Synthetic
IGWMTNNPPIPVGEIYKRWIILGLNKIVRMYSPTSILDIRQGPKEPFRDY

SEQ ID NO: 8 - protein, 9 aa, Artificial sequence
  Synthetic
NLVPMVATV

SEQ ID NO: 9 - protein, 9 aa, Artificial sequence
  Synthetic
KTWGQYWQV

SEQ ID NO: 10 - protein, 10 aa, Artificial sequence
  Synthetic
YLEPGPVTAK

SEQ ID NO: 11 - protein, 9 aa, Artificial sequence
  Synthetic
VLHDDLLEA

SEQ ID NO: 12 - protein, 9 aa, Artificial sequence
  Synthetic
RLRPGGKKK

SEQ ID NO: 13 - protein, 10 aa, Artificial sequence
  Synthetic
KRWIILGLNK

SEQ ID NO: 14 - DNA, 21 bp, Artificial sequence
  Synthetic
cagcagagaatggaaagtcaa

SEQ ID NO: 15 - DNA, 62 bp, Artificial sequence
  Synthetic
ctagacagcagagaatggaaagtcaactcgagttgactttccattctctgctgttttttgat

SEQ ID NO: 16 - DNA, 57 bp, Artificial sequence
  Synthetic
atcaaaaaaagcagagaatggaaagtcaactcgagttgactttccattctctgctgt

SEQ ID NO: 17 - DNA, 29 bp, Artificial sequence
  Synthetic
tggtcgctgctgtgatgtggaggaagaag

SEQ ID NO: 18 - DNA, 79 bp, Artificial sequence
  Synthetic
ctagatggtcgctgctgtgatgtggaggaagaagtcaagagcttcttcctccacatcacagcagcgaccattttttgat

SEQ ID NO: 19 - DNA, 75 bp, Artificial sequence
  Synthetic
atcaaaaaatggtcgctgctgtgatgtggaggaagaagctcttgacttcttcctccacatcacagcagcgaccat

SEQ ID NO: 20 - protein, 9 aa, Artificial sequence
  Synthetic
NLVPMVATV

SEQ ID NO: 21 - protein, 50 aa, Artificial sequence
  Synthetic
DIAGTTSTLQEQIGWMTNNPPIPVGEIYKRWIILGLNKIVRMYSPTSILD

SEQ ID NO: 22 - protein, 50 aa, Artificial sequence
  Synthetic
DIAGTTSNLQEQIAWMTNNPPIPVGEIYKRWIILGLNKIVRMYSPTSILD

SEQ ID NO: 23 - protein, 50 aa, Artificial sequence
  Synthetic
DIAGTTQTLQEQIGWMTNNPPIPVGEIYKRWIILGLNKIVRMYSPTSILD

(19) United States
(12) Patent Application Publication
(10) Pub. No.: US 2016/0045594 A1
(43) Pub. Date: Feb. 18, 2016

(54) DIRECTED IMMUNE STIMULATION

(71) Applicant: Fred Hutchinson Cancer Research Center, Seattle, WA (US)

(72) Inventors: Daniel E. Geraghty, Seattle, WA (US); Jodie P. Goodridge, Nesøya (NO)

(73) Assignee: Fred Hutchinson Cancer Research Center, Seattle, WA (US)

(21) Appl. No.: 14/780,379

(22) PCT Filed: Mar. 27, 2014
(86) PCT No.: PCT/US14/32091

§ 371 Date: Sep. 25, 2015

Related U.S. Application Data
(60) Provisional application No. 61/805,906, filed on Mar. 27, 2013.

(51) Int. Cl. A61K 39/385; C07K 16/28; C12N 7/00; A61K 39/21; A61K 39/00; A61K 39/245

(52) U.S. Cl. CPC A61K 39/385; A61K 39/0011; A61K 39/245; C12N 7/00; A61K 39/21; C07K 16/2833; A61K 2039/605; C12N 2710/16134; C12N 2740/16234; A61K 2039/57; C07K 2317/76; C07K 2317/622

(57) ABSTRACT

A synthetic polypeptide vaccine is employed for directed targeting of an individual's immune response to an antigen of interest. The synthetic polypeptide contains a carrier epitope specific for open conformers of an individual's MHC-I molecules in the presence of HLA-F, and also contains an effector epitope which elicits an immune response to the antigen of interest. The effect- or epitope may modulates an immune response to a tumor, pathogen, or autoantigen associated with an autoimmune disorder. The present vaccine exploits a role for the interaction between HLA-F and open conformers of MHC-I in the uptake of extracellular antigen for cross presentation.

### STATEMENT REGARDING FEDERALLY SPONSORED RESEARCH

This invention was made with government support in the form of grants P01 AI33484, RO1 HD45813, and CFAR P30 AI027757 from the United States Department of Health and Human Services, National Institutes of Health. The United States government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is: 43992_seq_final_{—}2014-03-27.txt. The file is 8 KB; was created on Mar. 27, 2014; and is being submitted via EFS-Web with the filing of the specification.

### BACKGROUND

It is well established that exogenous proteins are processed by professional antigen presenting cells (pAPC) in endosomes for MHC class II presentation to CD4+ cells while endogenous proteins are processed in the cytoplasm for MHC class I presentation to CD8+ T cells (Monaco, 1992 Immunol Today 13:173-179; York and Rock, 1996 Ann Rev Immunol 14: 369-396). Both intracellular transport and cell surface expression of MHC-I proteins are dependent on the availability of peptides within the endoplasmic reticulum, actively transported there by the transporter associated with antigen processing (TAP) complex (Peaper and Cresswell, 2008 Ann Rev Cell Dev Biol 24: 343-368). MHC-I can also present exogenous antigen by a process termed cross-presentation, which, as with MHC-II presentation, has been shown to operate primarily in pAPC (Amigorena and Savina, 2010 Curr Opin Immunol 22: 109-117). Many of the details of the specific pathways through which extracellular proteins are internalized and associate with MHC-I remain to be established (Bevan, 2006 Nature Immunol 7:363-365; Kurts et al., 2010 Nature Rev Immunol 10: 403-414). Attention has largely been focused on how peptides traffic to sites where MHC-I molecules reside during cross-presentation. Several models have been proposed for transport of peptides from extracellular derived compartments to the cytoplasm, including the ER-phagosome fusion model (Desjardins, 2003 Nature Rev Immunol 3:280-291) and the physical disruption of lysosomal membranes model (Luke et al., 2007 Cell 130: 1108-1119). Other studies have focused on pathways such as the ER-associated degradation (ERAD) pathway (Loureiro and Ploegh, 2006 Adv Immunol 92: 225-305), protein channels, and autophagy (Yeager and Harris, 2007 Curr Opin Cell Biol 19: 521-528). The uptake and cross-presentation of extracellular antigen may represent an important means to stimulate T cell responses to antigen otherwise not available through endogenous MHC presentation, as often occurs in tumor transformation or viral infection.

While the presentation of antigenic epitopes by classical MHC-I molecules is a critical component of the adaptive immune response, the human MHC also contains three nonclassical class I genes (HLA-E, F, and G) with divergent immune function. The HLA-E and G proteins evidently do not, at least as a primary function, participate directly in antigen presentation but rather interact with immunoregulatory receptors expressed on lymphocytes. HLA-G, expressed on placental trophoblast cells, may function as an important tolerogenic immunoregulator during pregnancy (Rajagopalan et al., 2006 PLoS Biol 4:e9; Shiroishi et al., 2006 Proc Natl Acad Sci USA 103: 16412-16417). HLA-E complex, expressed ubiquitously in coordination with classical MHC class I, interacts with the lectin heteroduplexes CD94 combined with different NKG2 subunits to inhibit and activate NK cells and subsets of T cells (Lee et al., 1998 Proc Natl Acad Sci USA 95: 5199-5204; Llano et al., 1998 Eur J Immunol 28: 2854-2863.).

HLA-F is expressed as a protein independent of bound peptide (Goodridge et al., 2010 J Immunol 184: 6199-6208; Wainwright et al., 2000 J Immunol 164: 319-328) and surface expression is upregulated in monocytes and most lymphocyte subsets upon activation, including Natural Killer (NK) cells, B cells, and all T cell subsets excepting regulatory T cells (Lee et al., 2010 Eur J Immunol 40: 2308-2318). MHC-I is also expressed on proliferating lymphoid cells as a stable pool of MHC-I heavy chain (HC) devoid of peptide and/or β2-microglobulin (β2 m) (Schnabl et al., 1990 J Exp Med 171: 1431-1442). These so called ‘open conformers’ have been implicated in a number of interactions with other receptors on the cell surface both in trans and in cis, including the formation of homodimers (Arosa et al., 2007 Trends Immunol 28: 115-123).

NK cells are an important component of the innate immunity that allow the immune system to respond to changes in class I major histocompatibility complex (MHC-I) expression, which can occur with tumor transformation or viral infection (Gumperz et al., 1996 J Exp Med. 183:1817-1827). Changes in MHC-I expression are sensed by polymorphic receptors on NK and T cells that are specific for allelic determinants on the MHC molecule itself (Lanier and Phillips, 1996 Immunol Today 17:86-91; Litwin et al., 1993 J Exp Med. 178:1321-1336). Among these, the killer cell immunoglobulin-like receptors (KIR) show the most developed ability to differentiate between different MHC allotypes (Parham et al., 2012 Philos Trans R Soc Lond B Biol Sci. 367:800-811), and are therefore of considerable interest for their involvement in transplantation (Cooley et al., 2010 Blood 116:2411-2419; Velardi et al., 2012 Curr Opin Hematol. 19:319-323), pregnancy (Male et al., 2010 Meth Mol Biol. 612:447-463), and infectious disease (Bashirova et al., 2011 Annu Rev Immunol 29:295-317). The genetic variation of KIR is extensive with respect to both gene content and allelic variation to a degree similar overall to that of MHC class I and II (Parham et al., id.; Uhrberg et al., 1997 Immunity 7:753-763). Of the better defined allo-specific receptor ligand pairs are the KIR2DL1 and KIR2DL2/3 and HLA-C group 1 and 2 (C1 and C2) receptor-ligand pairs defined by asparagine or lysine at position 80 of HLA-C alleles (Colonna et al., 1993 Proc Natl Acad Sci USA 90:12000-12004). Another is the KIR3DL1 mediated recognition of the Bw4 epitope present in a subset of HLA-A and -B alleles (Cella et al., 1994 J Exp Med. 180:1235-1242; Stern et al., 2008 Blood 112:708-710). Both sets of interactions function through an inhibitory activity for the respective KIR.

NK cells also interact with nonclassical class I MHC, including HLA-E and -G. Unlike classical MHC-I, HLA-G proteins do not function in classical antigen presentation but rather may function as an important tolerogenic immunoregulator during pregnancy through interactions with ILT2 and ILT4 and KIR2DL4 (Rajagopalan et al., 2006 PLoS Biol. 4:e9; Shiroishi et al., 2006 Proc Natl Acad Sci USA, 103:16412-16417; Shiroishi et al., 2003 Proc Natl Acad Sci USA 100:8856-8861). HLA-E, which apparently can present antigens to T cells in certain circumstances (Pietra et al., 2010 J Biomed Biotechnol. 2010:907092), primarily functions as a ligand for the CD94/NKG2 lectin receptors to regulate the activity of NK cells and subsets of NKT cells (Lee et al., 1998 Proc Natl Acad Sci USA 95:5199-5204; Llano et al., 1998 Eur J. Immunol 28:2854-2863). The human nonclassical class I HLA-F is expressed on proliferating lymphoid and monocyte cells as a molecule devoid of bound peptide with or without β2-microglobulin (β2 m) (Lee and Geraghty, 2003 J Immunol. 171:5264-5271). HLA-F appears to associate with other class I MHC proteins as open conformers without peptide (Goodridge et al., 2010 supra).

### SUMMARY

The present invention is directed to compositions and methods involving the non-classical HLA-F and open conformers of MHC-I expressed on activated cells in a pathway for the presentation of exogenous proteins by MHC-I. This pathway is distinguished from the conventional endogenous pathway by its independence from TAP and Tapasin and its sensitivity to inhibitors of lysosomal enzymes, and further distinguished by its dependence on MHC-I allotype-specific epitope recognition for antigen uptake. Thus, a previously unrecognized means of antigen cross-presentation mediated by HLA-F and MHC-I open conformers on activated lymphocytes and monocytes can be used by the compositions and methods provided herein to regulate immune system functions and an individual's immune defenses. Also, a broad interaction is identified between MHC-I open conformers—for which HLA-F is the prototypical example—and KIR receptors. This elucidates a more precise understanding of the interactions between MHC-I and KIR and consequent functions, and their targeting by immunomodulatory approaches to disease treatment and prevention and compositions described herein.

The present invention provides synthetic immunogenic polypeptides which bind to an open conformer of a Major Histocompatibility Complex (MHC) Class I molecule, e.g., HLA-B7 or B27, in the context of HLA-F. Fusion proteins and conjugates comprising at least one of the inventive immunogenic polypeptides described herein are also provided by the present invention. The present invention further provides nucleic acids encoding any of the inventive immunogenic polypeptides or fusion proteins described herein, expression vectors comprising nucleic acids encoding the synthetic polypeptides, and host cells comprising the vectors. Pharmaceutical compositions comprising any of the inventive synthetic immunomodulatory polypeptides, fusion proteins, conjugates, nucleic acids, expression vectors, host cells, or antibodies, and a pharmaceutically acceptable carrier, are provided herein. Also, vaccines comprising any of the inventive synthetic immunomodulatory polypeptides, fusion proteins, or conjugates are provided.

Thus, in one aspect of the invention a synthetic polypeptide vaccine is provided for targeting an individual's immune response to an antigen of interest. The polypeptide has an amino acid sequence which comprises a carrier epitope that binds to open conformers of the individual's MHC-I molecules, and an effector epitope which elicits an immune response to the antigen of interest. The carrier epitope is MHC-I HLA type specific for the individual. The polypeptide may be bounded by one or more caspase cleavage sites. The effector epitope modulates an immune response to the antigen of interest, such as a tumor, pathogen, or autoantigen associated with an autoimmune disorder. The synthetic vaccine may stimulate a CD8+ cytotoxic T cell response, and/or a CD4+ T helper cell response. The vaccine may further include an adjuvant or cytokine, including, for example, an agent which stimulates the expression of HLA-F.

When the synthetic polypeptide vaccine is directed to a tumor epitope, it may be derived from a tumor antigen of the tumor from the individual being vaccinated. The tumor antigen which is targeted may be identified in a proteome expression profile of the individual. In some instances the polypeptide may comprise more than one effector epitope which elicit immune responses to the same antigen, or which elicit immune responses to different antigens.

In some embodiments the synthetic polypeptide vaccine comprises an effector epitope that modulates an immune response to a minor histocompatibility autoantigen, as may be implicated in autoimmune disease, or even in adverse post-transplant reactions suffered by an individual.

The carrier epitope of the synthetic polypeptide vaccine binds to an open conformer of the individuals MHC-I molecule, preferably in the presence of HLA-F. The carrier epitope and the effector epitope may be the same sequence, from sequences of two different proteins from the same individual or two different individuals of the same species, or from proteins of different species.

In another aspect the invention provides a method for preparing an immunomodulating polypeptide that targets an individual's immune response to an antigen of interest. Such a method comprises determining the individual's MHC class I HLA type, selecting an amino acid sequence which comprises a carrier epitope that binds to MHC-I open conformers of the individual in the presence of HLA-F and is type specific for the individual, selecting an amino acid sequence which comprises an effector epitope of the antigen of interest to which immunomodulation is desired, and synthesizing the immunomodulating polypeptide which comprises the carrier epitope and the effector epitope. The immunomodulating polypeptide may contain compound HLA sites, and may contain an amino acid sequence for a marker or reporter epitope. When the individual's HLA MHC-II type is determined, the immunomodulating polypeptide may further comprise an epitope which binds the individual's MHC-II molecule. When the effector epitope is derived from a pathogenic virus, representative pathogens include herpes virus, retrovirus, hepatitis virus, or even a flavivirus. Specific examples of concern include human cytomegalovirus, hepatitis B or HIV. When the effector epitope is of a tumor antigen, it may be one from a solid tumor, a hematologic malignancy, or a melanoma. Among hematological malignancies are the group consisting of acute lymphoblastic leukemia, acute myelogenous leukemia, chronic lymphocytic leukemia, chronic myelogenous leukemia, Hodgkin's lymphoma, non-Hodgkin's lymphoma, and myeloma. In some instances an individual's tumor antigen is identified by proteome expression profiling of tumor cells obtained from the individual.

In other aspects the amino acid sequence of the carrier epitope can be altered to increase binding affinity of the polypeptide for the MHC-I open conformer of the individual. Or, for example, the amino acid sequence can be altered to decrease binding affinity of the polypeptide for the MHC-I open conformer of the individual, depending on the desired therapeutic or prophylactic application. The ability of the carrier epitope to bind the MHC-I open conformer can be determined on HLA-F positive cells, particularly those obtained from the individual. The immunomodulating polypeptide may comprise an epitope directed toward stimulating a CD4+ T helper response for the individual, and may comprises an epitope directed toward stimulating a CD8+ cytotoxic T cell response for the individual.

In another aspect a method is provided for directing the immune response of an individual to a target antigen of interest. This aspect comprises determining the individual's MHC class I HLA type, and contacting cells of the individual that have been upregulated for HLA-F expression upon receiving an activating agent, with a synthetic immunomodulating polypeptide which comprises a carrier epitope that binds to open conformers of the individual's MHC-I molecules and an effector epitope which elicits an immune response to the antigen for which immunomodulation is desired, thereby directing the immune response of the individual to said antigen of interest. The cells that upregulate HLA-F expression are lymphocyte, monocytes, dendritic cells and epithelial cells, among others. The contacting step may be performed ex vivo, and further comprising the step of returning the cells to the individual, or in vivo. The cells can be activated to express HLA-F by treatment with CD40 ligand, adjuvant, human TLR ligand (e.g., TLR9 ligand), or TNF-alpha and interferon gamma.

In yet another aspect, the invention provides a method for down-regulating the immune response of an individual to an antigen. This aspect comprises determining the individual's MHC class I HLA type, and contacting cells of the individual that have been upregulated for HLA-F expression with an inhibitory agent which specifically inhibits the expression of HLA-F on the surface of the individual's activated, thereby down-regulating the individual's immune response to the antigen. The inhibitory agent which inhibits the expression of HLA-F can be a monoclonal antibody or binding fragment thereof that specifically binds to the HLA-F heavy chain, e.g., to an extracellular domain of the HLA-F heavy chain. The antibody can be a single chain antibody, or a binding fragment thereof. The immune response to be down-regulated may be an inflammatory response.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows that exogenous antigen down-modulates HLA-F surface expression. (A) HLA-F surface expression is down modulated by addition of denatured full-length protein. HLA-F surface expression on B, T, or monocyte cell lines was measured before (solid line) or after (dotted line) addition of denatured recombinant HCMV pp65 protein or HIV-1 p24 protein. For all stains, HLA-F was measured with mAb 3D11 with isotype matched irrelevant antibody (gray). HLA-F and MHC-I co-localize with exogenously added protein on the surface and internally. B-LCL BM9 cells were stained with 3D11 or MHC-I HC mAb HCA2 and biotinylated gp100 50-aa protein or HA-1H 50-aa protein as indicated above each cell profile. Surfaces were stained with W6/32 and nuclei were stained with DAPI. The results of each set of stains are merged on the far right as indicated. (B) MHC-I complex formation on T2 cells (TAP and Tapasin deficient) was used as a measure of exogenous antigen uptake. The increase in surface MHC-I complexed with peptide and β2m was measured using mAbs W6/32 and mA2.1 before and after addition of exogenous pp65 protein and peptide. The change in mean fluorescence index (MFI) using the indicated antibodies was measured (average of three independent measurements) with and without the addition of inhibitors as indicated in the legend between the graphs. (C) Partially processed exogenous pp65 antigen co-localized in cellular fractions containing early endosomes and further processed proteins co-localized with fractions containing lysosomes. Western analysis was performed with mAb indicated to the left of each gel profile with molecular weight markers to the right. Gradient fraction number is indicated above each lane and relative enzyme activity measured within each fraction is indicated beneath the gels.

FIG. 2 shows epitope-specific binding of 50-amino acid polypeptides to MHC-I open conformers. (A) HLA-A*02 binding peptides differentially up-regulate MHC-I complex and down-regulate HLA-F and MHC-I open conformer. Normalized mean florescent intensity (MFI) is plotted for each of the indicated mAb stainings of T2 with and without the addition of the indicated peptides. (B) Higher affinity of peptide sequences is mirrored in stronger binding of 50-aa polypeptides to T2 cells. Plotted are MFIs of T2 cells stained with 50-aa biotinylated proteins containing the respective low (ELE), medium (YLE), and high (YLF) affinity epitope sequences as defined by direct peptide binding. Binding of 50-aa biotinylated proteins is reduced by prior addition of anti-MHC-I open conformer mAbs HC10 and HCA2 on both T2 cells and HLA-A*02 expressing LCL HOM2 as indicated. (C) MHC-I and HLA-F specifically interact with 50-aa polypeptides and relative binding reflects the peptide epitope affinity. Quantitative precipitation of MHC-I and HLA-F protein with 50-aa polypeptides containing the low affinity and high affinity peptide sequences reflects their surface binding affinity. T2 and T2/B35 transfectants were incubated with the indicated biotinylated 50-aa polypeptides and protein was precipitated as described in Methods. Western blot analysis of the fractionated precipitates is shown using HCA2 (MHC-I) and 3D11 (HLA-F).

FIG. 3. (A) Sensitization of B-LCL targets by exogenous protein. Specific CTL clone response was induced upon exposure of B-LCL to exogenous proteins. Each profile presents data using a CTL clone specific for a known epitope within either HCMV protein pp65 or HIV-1 p17 sequence. The effector name, epitope, and MHC-I restrictions and the target names and expressed HLA alleles are indicated immediately above each profile. A unique legend is presented in the upper right corner of each profile. For pp65, antigen was delivered either as peptide, endogenously via vaccinia construct, or exogenously as denatured recombinant protein. The p17 antigen was delivered as 50 amino acid protein containing the respective CTL epitopes and nonamer peptides were used as positive and negative controls as appropriate for each effector Immediately to the right of each profile are bar graphs reporting sensitization of the corresponding targets to CTL lysis in the presence or absence of Brefeldin A (BfA). Specific lysis at a single E:T ratio was measured without (solid) or with (hatched) prior treatment of targets with BfA for each peptide and the respective proteins as indicated. Antigen presentation from exogenous protein can occur in T cell and monocyte cell lines. (B) Adding denatured proteins exogenously to corresponding CTL clone cultures elicits IFN-G production. Fluorescence measurements of IFN-G are plotted versus forward scatter after the addition of either exogenous viral or minor histocompatibility antigen protein to the respective CTL clones as indicated. Each negative control experiment was performed in parallel cultures under identical conditions without the addition of peptide (pep) or protein. Numbers within each quadrant record the percentages of the total contained within the respective quadrant.

FIG. 4 shows the specific HLA-F shRNA knockdown constructs interfere with surface binding of antigen and exogenous antigen-derived target sensitization. (A) FACS analysis of B-LCL KOSE infected with shRNA construct F4 targeting HLA-F (solid line) or shRNA vector only (dotted line) with isotype matched control mAb (gray line). Each pair of profiles was analyzed with anti-HLA-F (mAb 3D11) and anti-MHC-I HC (mAb HCA2) as indicated. (B) Surface binding of proteins is reduced coincident with reduced levels of HLA-F and MHC-I. Mean florescence index measured on B-LCL KOSE and corresponding transfectants was plotted versus increasing concentrations of biotinylated 50-aa proteins gp100#2 or HA-1H as indicated. (C) Sensitization of KOSE transfectant targets by HCMV derived pp65 antigens. T cell clone 1C7-31 restricted by HLA-A*0201 was used as effector in the experiments against the 3 transfected targets after the addition of peptide, denatured recombinant pp65, or after infection with recombinant vaccinia producing pp65 as indicated above each panel. Specific lysis is plotted versus E:T ratio for each of 4 targets as indicated in the legend in the first panel. Control, which is replicated in each panel, was KOSE as target without the addition of any form of pp65 antigen. (D) mAb specific for HLA-F and MHC-I HC inhibit lysis of antigen-pulsed B-LCL 721 by pp65 specific CTL. Increasing amounts of denatured pp65 protein were added to cells incubated with the indicated mAbs (see legend) and tested for specific lysis with effector 1C7-31.

FIG. 5 shows antigen presentation through the HLA-F associated pathway is independent of TAP or Tapasin. (A) Endogenous HA-1H antigen presentation is TAP dependent. Specific CTL clone response to endogenous antigen was tested upon exposure to B-LCLs 721 and derivative LCLs 0.134 (TAP negative), 0.134C2 (0.134 TAP transfectant restored), and 0.174 (MHC class II region deletion, including TAP and Tapasin). (B) Exogenous HA-1H antigen is processed and presented in TAP and Tapasin negative cells. Both 0.134 and T2 (0.174 chr 6) are sensitized to lysis by the HA-1H specific CTL clone upon addition of a 50 amino acid (50-aa) protein containing the HA-1H epitope, but not a similar 50-aa protein representing the alternative HA-1R allele. HA-1H nonamer peptide was included as positive control and parallel analysis without added peptide served as negative control (see legend). (C) TAP and Tapasin negative cells pulsed with exogenous pp65 protein, but not endogenous pp65 protein, are efficiently lysed by specific CTL clone. HCMV pp65 protein was delivered endogenously via vaccinia construct or exogenously as denatured recombinant protein, with peptide controls as indicated. For each set of experiments (A-C) targets are indicated above each profile and CTL clone effectors and MHC-I specificities are indicated at the top each set of profiles. Legends are provided at the upper right corner of the first of each set of profiles. Bar graphs immediately to the right of each profile report results from a single E:T ratio of the corresponding effector and target for peptide or protein as indicated beneath the bars without (solid bar) or with (hatched bar) the prior addition of BfA.

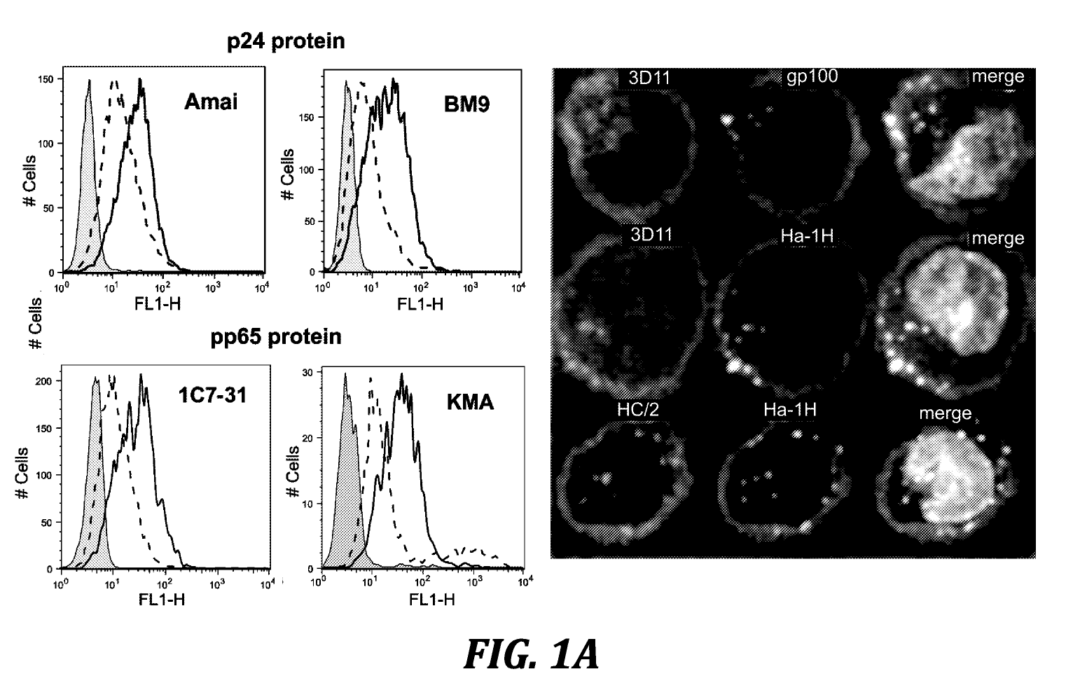

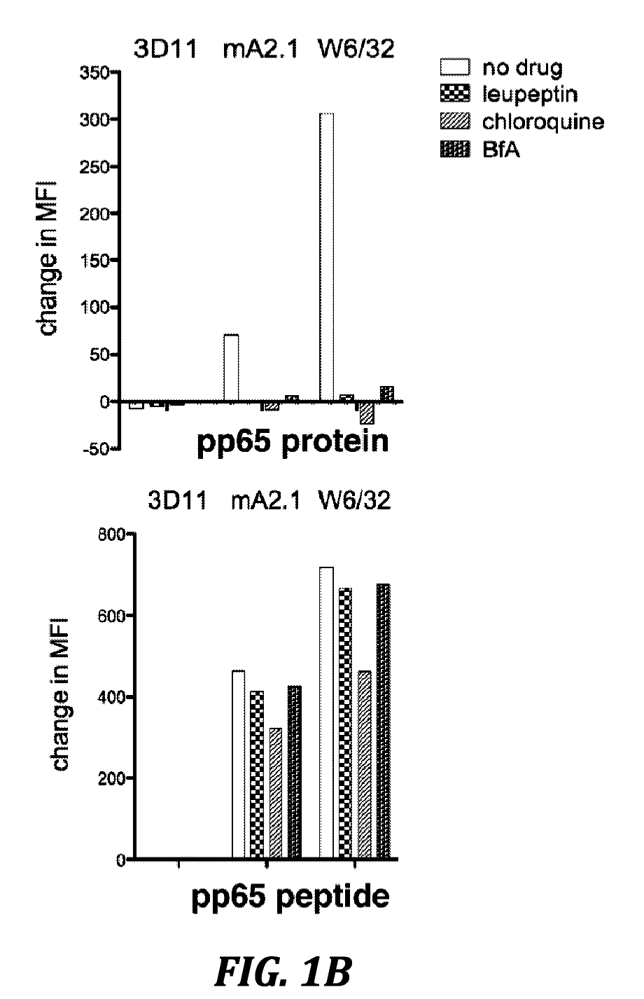

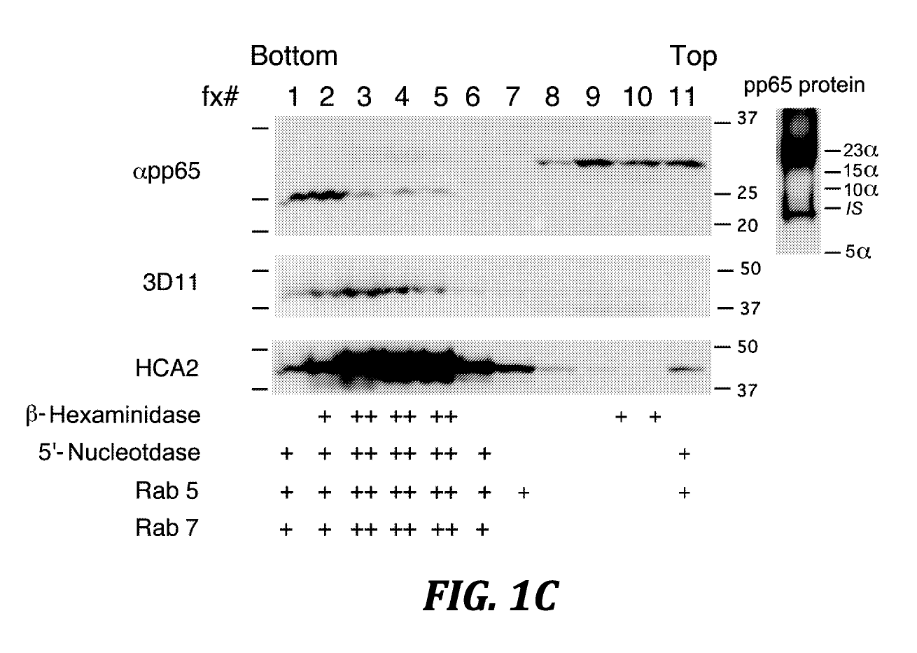

Figure S1 shows the peptide titration experimental results used as a basis for the results reported in FIG. 1B. Peptide titration on T2 cells. T2 cells were analyzed by FACS using the indicated mAbs (see legend) before and after incubation with increasing concentrations of pp65 peptide, and the change in mean florescence index (MFI) is plotted versus peptide concentration.

Figures S2 and S3 include results from control experiments demonstrating that sensitization to lysis by whole protein in experiments reported in the paper were not due to contaminating peptide. Figure S2 displays the results from LC-MS/MS experiments conducted to rule out the presence of preexisting peptide in protein preparations. It also includes the results of mock incubation experiments that demonstrate peptide epitopes were not generated artifactually outside of target cells during experimental incubations. Sensitization to lysis by whole protein is not due to contaminating peptide. (A) Protein preparations are free of peptide at levels above minimal concentrations effective to sensitize targets. Titrations of specific peptide and corresponding proteins were carried out in Cr release assays and the minimal peptide and protein concentrations that elicited 40%-maximal specific lysis are reported in the peptide conc. and protein conc. columns respectively. The ratio of these numbers is listed in the Pep:pro ratio column. Each protein was examined for the presence of peptide by MS/MS and the minimal detectable amount of peptide is reported/the quantity of protein examined in each experiment yields the limits of detection in the respective quantity of protein (as a molar ratio calculating peptides at 1 kD and proteins at 5kD excepting pp65 at 50kD) Minimum detection limits were established using direct analysis of each specific peptide, and ranged from 1 pg (p24) to 100 pg (p17) for the five proteins tested and the indicated amounts of protein were analyzed by LC-MS/MS with no detectable peptide. By combining these data it was apparent that preexisting peptide was not detectable in any protein preparations to levels of from ten to two hundred-fold below the minimum required to effect target lysis, with the exception of pp65. For pp65 no peptides were detected, but the lower detection limit was greater than the minimum required for 40% target lysis. (B) A sample spectra of 10 pg of each peptide is shown, excluding p17 peptide [SEQ ID NO:1] KIRLRPGGKKK which was only detectable at 100 pg. (C) Prior incubation of proteins with targets does not spontaneously generate specific peptide released into culture. A single E:T ratio was tested for each clone titrating peptide and protein concentrations as indicated. Peptide or protein were incubated with targets as described in Methods for the appropriate times, up to the point of exposing targets to effectors, supernatants were collected and portions spun through centricon membranes with cutoff sizes permissible for peptide pass-through. These two preparations, before and after pass-through, were then used to sensitize targets as before in titrations spanning the effective limits of sensitization. For protein, but not for peptide, the centricon eliminated sensitization to lysis consistent with the absence of specific peptide being generated during incubation of proteins with targets.

Figure S3 presents evidence against preexisting peptide and against artifactual generation of peptide from experiments using a temperature difference to distinguish between sensitization of targets by peptide and protein. Reduced temperature lowers surface binding of exogenous protein coincident with reduced levels of antigen presentation. (A) HLA-F surface expression and surface binding of p17 and p24 50 amino acid (50-aa) proteins were measured at 4° C. (dotted lines) and 37° C. (solid lines). N-terminal biotinylated p17 and p24 proteins were detected with streptavidin-PE. (B) Specific lysis of targets SAVC and HOM2 sensitized with increasing concentrations of exogenous peptide or protein at 4° C. or 37° C. by effectors SR01 (p17 RLR) and 7709 (p24 RKW) as indicated in the legend above each graph.

### DETAILED DESCRIPTION

While the preferred embodiment of the invention has been illustrated and described, it will be appreciated that various changes can be made therein without departing from the spirit and scope of the invention.

The present invention exploits a role for the interaction between HLA-F and open conformers of MHC-I in the uptake of extracellular antigen for cross presentation.

Several lines of evidence support an active role for open conformers of MHC-I in the uptake of antigen as whole or partial protein that contains epitopes specific to receptive MHC-I expressed on target cells. Exogenous antigen is internalized together with MHC-I, and down-modulation of surface MHC-I results in reduced binding, uptake and presentation of antigen. Differential binding of polypeptides containing low and high affinity MHC-I binding epitopes to MHC-I and HLA-F indicates that exogenous antigen binds to the surface of activated cells to a structure that includes MHC-I OC and HLA-F and that is in contact with the epitope sequence within the extended polypeptide. The need for exposure of linear epitopes is consistent with the observation that protein required denaturation in order to enter this pathway. Although offered by way of possible explanation and not intended as a limitation of the invention, MHC-I open conformer, stabilized by HLA-F, may retain rudimentary peptide binding specificity conferred by the peptide binding cleft and can bind epitopes in an open ended fashion without size limitations, possibly in a manner similar to MHC class II.

Based on many crystal structures and considerable binding and T cell recognition data, a folded class I MHC molecule binds 8-10 mer peptides, depending on the allele and epitope, and that in contrast to MHC-II, the ends of the pocket are closed (Peaper and Cresswell, supra). There are few reported exceptions to this rule. The discovery as part of the present invention is that extended polypeptide chains are capable of binding to MHC-I open conformers. The structure of the MHC-I open conformer is unknown and is most certainly distinct from the classical structure consisting of HC, β2m, and peptide. Determining the MHC-I open conformer structure could address possibilities for an alternative antigen binding mechanism related to the presently described interaction of the MHC-I binding cleft with epitopes within extended polypeptides, i.e., those of at least about 30, 40, 50, 60, 70, 80 and more amino acids, for example from 100 to 150, up to 200 amino acids, and even up to 300, 400 and 500 amino acids in the extended polypeptide can be used according to the present invention.

In addition to MHC-I OC, several experiments described in detail below directly implicate HLA-F in this pathway. First, the observed overlapping internalization and localization of antigen and HLA-F was coincident with the same observation with MHC-I OCs. Secondly, HLA-F was co-precipitated with MHC-I by polypeptide containing MHC-I binding epitopes at relative levels roughly paralleling those of MHC-I. Third, down modulation of HLA-F resulted in interference with antigen binding, uptake, and processing for presentation, and although down modulation was coincident with down modulation of MHC-I, blocking with HLA-F specific mAb alone interfered with antigen cross-presentation. One role for HLA-F may be in the stabilization and transport of MHC-I open conformer to, on, and from the surface. The physical interaction between HLA-F and classical MHC-I OC, their coincident surface expression on activated lymphocytes, and their coincident down modulation in HLA-F knockdowns combine to suggest that they are interdependent for surface transport. HLA-F may stabilize MHC-I OC as it is formed, and the two proteins, possibly as a heterodimer, transit to the surface. Other not mutually exclusive roles include cooperation in the internalization of antigen and MHC-I.

The evidence presented as part of the present invention supports that antigen, MHC-I open conformer, and possibly HLA-F transit from the surface through the endosomal pathway into lysosomes or lysosome-like structures where protein is degraded to produce target peptide independently of TAP or Tapasin. After complex formation, MHC-I containing specific peptide derived from the exogenous antigen source is transported to the surface. This proposed mechanism has similarities with the MHC-II antigen presenting pathway. There is evidence that MHC-I molecules visit phagolysosomal compartments to acquire peptides prior to surface expression (Bachmann et al., 1995 Eur J Immunol 25: 1739-1743; Gromme et al., 1999 Proc Natl Acad Sci USA 96: 10326-10331). MHC-I proteins have been shown to reside in endosomes and lysosomes of dendritic cells and exchange of MHC-I between the cell membrane and endosomal compartments has been demonstrated in both T cells and macrophages (Kleijmeer et al., 2001 Traffic 2: 124-137; MacAry et al., 2001 Proc Natl Acad Sci USA 98: 3982-3987; Reid and Watts, 1990 Nature 346: 655-657). Further, in the mouse, exogenous MHC-I antigen loading has been associated with endosomal and lysosomal trafficking in dendritic cells (Basha et al., 2008 PLoS One 3: e3247).

While previous studies have provided evidence in support of a pathway for class I loading that is shared with class II molecules (e.g., Gromme et al., 1999, supra), the present invention provides at least three major developments. First, no evidence implicating the participation of MHC-I open conformer, nor any including HLA-F has been reported. Second, no studies or proposed models have suggested an allelic dependence on MHC-I specific epitopes for antigen uptake. Third, most cross presentation pathways studied were operating in professional antigen presenting cells, primarily dendritic cells (Ackerman and Cresswell, 2004 Nature Immunol 5: 678-684; Basta and Alatery, 2007 Scand J Immunol 65: 311-319; Guermonprez and Amigorena, 2005 Springer Semin Immunopathol 26: 257-271; Randolph et al., 2008 Curr Opin Immunol 20: 52-60; Shen and Rock, 2006 Curr Opin Immunol 18: 85-91), rather than nonprofessional antigen presenting cells. It has been shown that exogenous recombinant influenza A virus nucleoprotein is processed and presented via MHC-I by EBV-transformed B-LCLs (Voeten et al., 2001 Clin Exp Immunol 125: 423-431). Also, not only dendritic cells and monocytes but also B cells can cross-present uBZLF1 in vitro (Barabas et al., 2008 PloS Pathog. 4: e1000198).

Perhaps because relatively few examples of cross-presentation have been suggested as acting in nonprofessional antigen presenting cells, the advantages that cross presentation in amateur APC offer the host has not been apparent. Given a requirement for HLA-F and MHC-I open conformers, this mechanism would necessarily be restricted to activated cells, which benefit from the ability to take up and process extracellular antigen for a number of different reasons. For example, the possibility that monocyte-derived cells can participate critically in processing antigen for cross-presentation has been suggested, even if they do not present that antigen to T cells themselves (Randolph et al., 2008 supra). Also, cross-presentation by nonprofessional APCs has been demonstrated for HLA class I epitopes from exogenous NY-ESO-1 polypeptides (Gnjatic et al., 2003 J Immunol 170: 1191-1196). Membrane transfer of antigens from activated B cells to bystander B cells was recently demonstrated (Quah et al., 2008 Proc Natl Acad Sci USA 105: 4259-4264). Transfer from other activated cells could also be explained by the process of intercellular communication of biomolecules through exosomes (Cocucci et al., 2009, Trends Cell Biol 19:43-51). This points to the possibility of transfer of lipid rafts containing processed antigen, presented by MHC-I from activated NK or T cells to monocytes, dendritic cells, or B cells (Cocucci et al.; Luketic et al., 2007 J Immunol 179: 5024-5032; Mignot et al., 2006 J Cell Mol Med 10:376-388; Segura et al., 2007 J Immunol 179:1489-1496). Another explanation includes NK cells acquiring antigen from target cells for subsequent stimulation of T cells (in this case memory T cells), particularly when a target cells is lacking in MHC expression. Very little detail has been described regarding how memory CD8+ T cells are activated, including their potential activation by amateur-APCs (Yewdell and Haeryfar, 2005 Ann Rev Immunol 23: 651-682).

A cross-presentation immune pathway may play a role in protection from pathogens (Basta and Alatery, 2007, supra; Shen and Rock, 2006 supra; Vyas et al., 2008 Nature Rev Immunol 8: 607-618). Thus, with MHC-I open conformers and HLA-F functioning in MHC-I antigen cross presentation, antigen uptake, processing, and presentation particular to this pathway, as described herein, new approaches are provided by the invention for the design and optimization of immunomodulating agents.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. As used in this specification, the term “about” refers to a range of slight variation, such as 10%, above or below the stated figure.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Unless specifically defined herein, all terms used herein have the same meaning as they would to one skilled in the art of the present invention. Practitioners are particularly directed to Sambrook, et al., Molecular Cloning: A Laboratory Manual, 3d ed., Cold Spring Harbor Press, Plainsview, New York (2000) and Ausubel, et al., Current Protocols in Molecular Biology (Supplement 47), John Wiley & Sons, New York (1999), which are incorporated herein by reference, for definitions and terms of the art.

The present invention provides methods to design and select polypeptides for use in stimulating specific immune responses, and also provides immunogenic vaccine compositions and formulations of said polypeptides. Typically the polypeptides are from about 27 or more amino acids, more typically at least about 30 amino acids, 30-50 amino acids, more typically at least about 30, 40, 50, 60, 70, 80 and more amino acids, for example from 100 to 150, up to 200 amino acids, and even up to 300, 400 and 500 amino acids in the extended polypeptide can be used according to the present invention, depending on, e.g., additional epitopes targeted, sequences to be used as markers, etc. The design described herein of immunomodulating polypeptides uses knowledge of MHC-I peptide epitope binding and of HLA-F expression to specifically enhance directed immune responses. An approach for immunization is provided by the instant invention that takes into account: (i) an individual's HLA type, (ii) the specific epitopes for which targeting immunization is desired, whether they be tumor antigens, autoimmune antigens, viral antigens, or other targets; (iii) the ability to increase uptake and processing of antigen using attached high affinity epitopes possibly unrelated to the targeting epitope, (iv) the ability to control or enhance a specific cellular pathway for stimulation (e.g. CD4 vs CD8 T cells) through polypeptide design, (v) the ability to control the cell type to target for initial immunization based on activation state.

Previous methods for immunization employ polypeptides constructed without direct knowledge of an individual polypeptide's capacity to provoke a particular immune response. In contrast, the vaccines and methods of the present invention allow for responses to be directed at specific branches of the immune response and at particular epitopes suitable for an individual's MHC type. The present invention allows for a design of polypeptides that will enhance their uptake by immune cells and processing and presentation to other immune cells. Thus, synthetic long polypeptides can be designed to bind “open” class I MHC and/or HLA-F on activated HLA-F positive cells. Long polypeptides bound to class I MHC/HLA-F are incorporated into the cell and presented as class I MHC complexes capable of stimulating specific T cell responses to cancer and viral infection. Binding of long polypeptides is affected by the sequence in such a way that mutation of positions within class I epitopes encoded within the polypeptide sequence can be used to increase or decrease binding and consequent responsiveness to the antigen. In this way, polypeptides can be designed using a carrier epitope that affects binding to cells, combined with an effector epitope for the target antigen. In light of this, it is also possible to include reporter epitopes to known antigens with which to standardize assays and responses. In addition, induction of HLA-F expression in tumors and tumor infiltrating lymphocytes, and lymph nodes contribute to the efficacy of the present invention. Further, polypeptides are used to specifically interfere with target antigen uptake via this pathway in methods such as cross presentation of minor histocompatibility antigen post-transplant, (pro)insulin and Beta cells antigen in development of diabetes and other autoimmune disorders as discussed below.

As used herein, the term “synthetic polypeptide” means a poly amino acid molecule of at least about 27 amino acids that is not naturally occurring. The synthetic polypeptide can be made by any known method available in the art. For instance, the synthetic polypeptide can be synthesized by the iterative addition of each amino acid in sequence according to standard solution or solid phase synthesis techniques to ultimately form the complete synthetic polypeptide that is contemplated. Alternatively, the synthetic polypeptide can be produced recombinantly according to standard techniques. For example, as is well-understood, a DNA template construct can be created that contains one or more sequences from the same or different sources genetic sources. The DNA template is introduced into an organism capable of transcribing the template and ultimately expressing the synthetic polypeptide using its cellular expression components. The synthetic polypeptide describe herein comprises one or more carrier epitopes.

As used herein, the term “carrier epitope” means any domain of the synthetic polypeptide that can bind to open conformers of an individual's MHC I molecule. The carrier epitope is at least 8 to 10 amino acids in length. An open conformer is a domain of an expressed MHC I molecule that is devoid of bound peptide and/or β2-microglobulin. MHC I open conformers on the cell surface are typically associated with HLA-F molecules. In some embodiments, the synthetic polypeptide comprises a plurality of carrier epitopes. Two or more of the carrier epitopes can have the same or substantially similar sequence. In some embodiments, two or more carrier epitopes can bind to the same open conformer. In other embodiments, the two or more carrier epitopes can bind to distinct open conformers. As an important event in the MHC class I epitope presentation pathway is the binding to the MHC molecule, much work has been done to predict MHC binding of peptides (epitopes), as explained in detail in, e.g., Lundegaard et al., J Immunol Methods 2011 374(1-2): 26-34, which is expressly incorporated herein by reference for that purpose. The sequence of the carrier epitope can be optimized for binding the MHC-I open conformer, either a higher binding affinity or lower binding affinity, as may be desired for the particular application described herein. Such sequence optimization procedures are generally known, see, e.g., U.S. Pat. No. 8,614,304, which is incorporated herein by reference.

The synthetic polypeptide described herein also comprises an effector epitope. As used herein, the term “effector epitope” means any domain of the synthetic polypeptide that comprises a linear polypeptide (“effector polypeptide”) sequence that, upon internal cellular processing of the effector epitope, is loaded onto the MHC molecule and expressed in such association on the surface of the cell, and capable of stimulating a response by an immune cells, e.g., upon binding of the immune cell's T-cell receptor (TCR) to the MHC/effector polypeptide complex. The effector polypeptide can be the same as, or a subcomponent of the effector epitope. As is generally understood, the internal cellular processing of the effector polypeptide can comprise digestion of the effector epitope or sequence adjacent to the effector polypeptide, transport of the effector polypeptide into the endoplasmic reticulum by the transporter associated with antigen processing (TAP). TAP forms a multimeric complex with various other molecules that stabilizes the MHC molecule and loads the effector polypeptide onto the MHC molecule. The MHC/effector polypeptide complex travels via the secretory pathway to the cell membrane.

In some embodiments, the synthetic polypeptide comprises a plurality (i.e., one or more) effector epitopes. In some embodiments, two or more of the plurality of effector epitopes have the same or substantially similar sequences. In some embodiments, two or more of the plurality of effector epitopes are different yet derived from the same antigen. In some embodiments, two or more of the plurality of effector epitopes are from distinct antigens derived from the same tumor or pathogenic agent.

In some embodiments, the carrier epitope and the effector epitope are the same. In other embodiments, the carrier epitope and the effector epitope overlap. In yet other embodiments, the carrier epitope and the effector epitope are distinct domains of the synthetic polypeptide. The distinct domains can be immediately adjacent within the synthetic polypeptide or separated by any number of intervening amino acid residues. Such intervening amino acid residues can serve as additional domains of the synthetic polypeptide. In some embodiments, the synthetic polypeptide is a fusion polypeptide comprising distinct domains (e.g., the carrier epitope and effector epitope). The synthetic polypeptide may be bounded (carboxyl and amino termini) by caspase cleavage sites. For a polypeptide derived from a naturally occurring protein, such sites likely define the polypeptides that will occur in vivo when cells apoptose or equivalent—then caspases are released, digest proteins, resulting in polypeptides that can be taken up via the HLA-F system described herein. In some embodiments, the carrier epitope and effector epitope are derived from the same native protein, but appear in the fusion polypeptide in an orientation or configuration relative to each other that is not provided in the native protein. In some embodiments, the carrier epitope and effector epitope are derived from different alleles of the same native protein an organism or species of organism. In some embodiments, the carrier epitope and effector epitope are derived from different native proteins. In some embodiments, the carrier epitope and effector epitope are derived from different proteins an organism or species of organism. In yet other embodiments, the carrier epitope and effector epitope are derived from proteins of different species of organism. Stated alternatively, the carrier epitope and effector epitope are heterologous. It will be appreciated that the term “fusion” as used in this context implies only the non-natural relationship or relative configuration of the multiple domains, and does not necessarily imply the procedure for creating the synthetic polypeptide.

In some embodiments, the synthetic polypeptide further comprises one or more MHC II antigen sequences than can be the same as, overlap with, or distinct from, any of the domains described above.

As used herein, the term “antibody-like molecule” encompasses antibodies or fragments thereof. Unless otherwise stated, exemplary antibody-like molecule can include antibodies such as monoclonal, multispecific antibodies (e.g., bispecific antibodies), chimeric antibodies, anti-idiotype antibodies, and may be any intact molecule or fragment thereof, and of any isotype. Also, the antibody-like molecule encompasses molecules that comprise any of the foregoing, such as fusion proteins.

As used herein, the term “antibody fragment” refers to a portion derived from or related to a full-length antibody, generally including the antigen binding or variable region thereof. Illustrative examples of antibody fragments include Fab, Fab′, F(ab)2, F(ab′)2 and Fv fragments, scFv fragments, diabodies, linear antibodies, single-chain antibody molecules and multispecific antibodies formed from antibody fragments.

As used herein, the term “single-chain antibody” refers to an antibody fragment that contains at least one antigen binding region in a single polypeptide molecule. For example, as used herein, the term “single-chain Fv” or “scFv” specifically refers to an antibody fragment that comprises the VH and VL domains of an antibody, wherein these domains are present in a single polypeptide chain. Additionally, the Fv polypeptide further comprises a polypeptide linker between the VH and VL domains, which enables the scFv to form the desired structure for antigen binding. An scFv can also be generated to be multivalent, namely to contain multiple pairings of VH and VL domains in a single polypeptide chain, where each pairing can bind to the same or different antigen.

The disclosed synthetic polypeptide vaccine compositions and methods can be applied to address infection of the individual by any known pathogen. To address such infections, the compositions and methods of the present disclosure can incorporate antigenic epitopes from any known pathogen to assist the stimulation of an immune response thereto. A pathogen is any heterologous (e.g., non-self) biological entity that infects a subject and can produce a disease therein. Pathogens are typically infectious agents that can be categorized as subcellular (e.g., prionic or viral), prokaryotic (e.g., bacterial), fungal, or multicellular (e.g., eukaryotic and/or parasitic).

For example, prionic pathogens typically do not contain nucleic acids, but are abnormally formed proteins that can affect folding of host proteins. Known prions are involved in diseases such as scrapie, bovine spongiform encephalopathy (mad cow disease) and Creutzfeldt-Jakob disease.

Many viruses are known to be associated with disease and are, therefore, applicable to the present disclosure. For example, viruses typically associated with disease can be selected from among the following viral families: Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, Togaviridae, and the like. Illustrative, non-limiting examples of clinically important viruses include Adenovirus, Herpes simplex, type 1, Herpes simplex, type 2, Varicella-zoster virus, Epstein-barr virus, Human cytomegalovirus, Human herpesvirus, type 8, Human papillomavirus, BK virus, JC virus, Smallpox, Hepatitis A virus, Hepatitis B virus, Human bocavirus, Parvovirus B19, Human astrovirus, Norwalk virus, coxsackievirus, poliovirus, rhinovirus, Severe acute respiratory syndrome virus, Hepatitis C virus, yellow fever virus, dengue virus, West Nile virus, Rubella virus, Hepatitis E virus, Human immunodeficiency virus (HIV), Influenza virus, Guanarito virus, Junin virus, Lassa virus, Machupo virus, Sabiá virus, Crimean-Congo hemorrhagic fever virus, Rabies virus, Hepatitis D, Rotavirus, Orbivirus, Coltivirus, Banna virus, and the like.

Similarly, many bacteria are known to be associated with diseases and are, therefore, applicable to the present disclosure. Such bacteria can infect a subject host in either the intracellular (e.g., Chlamydophila, Ehrlichia, Rickettsia) or extracellular space (e.g., Staphylococcus, Streptococcus, Pneumococcus, etc.). For example, bacteria typically associated with disease can be selected from among the following genera: Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, Yersinia, and the like.

Similarly, many eukaryotic organisms are known to be associated with diseases and are, therefore, applicable to the present disclosure. Such known eukaryotic pathogens (parasites) can be any single-celled protozoan pathogens or multicellular parasites that are known to infect human and other host subjects. Illustrative, non-limiting protozoan pathogens include the Kinetoplasta (e.g., trypanosomes, leishmanias), Giardia, Trichomonas, amebas, Apicomplexa (e.g., Toxoplasma, Cyclospora, Plasmodium, and the like). Illustrative, non-limiting multicellular parasites include platyhelmiths (flatworm) parasites (e.g., trematodes and cestodes), and nematode (roundworm) parasites (e.g., Tricuris, Trichinella, Ascaris, Enterobius, and the like).

The disclosed methods and compositions can be applied to address neoplastic conditions and cancers of the subject. To address such conditions, the compositions and methods of the present disclosure can incorporate antigenic epitopes associated with any transformed neoplastic or cancer cell. In this regard, transformed neoplastic or cancer cells are endogenous cells that are abnormally proliferative and tend to acquire abnormal characteristics not in common with the cell-type from which they derived. For example, such transformed cells make start expressing (or increase expression) of particular proteins not commonly expressed in normal cells. Such proteins can serve as epitopes that are associated with the cancer cells and can be useful in the present disclosure to stimulate an immune response against such transformed cells. In this context, the use of the term cancer cells refers to cells that exhibit unregulated or uncontrolled cell division, many of which are well characterized in the art. Cancer cells can be cells residing in the body of an animal subject. Potential cancer types include carcinomas (derived from epithelial cells), sarcomas (derived from connective tissue, or mesenchymal cells), lymphoma and leukemias (derived from hematopoietic cells), blastomas (which are derived from immature or embryonic cells), or germ cell cancers. Among the hematological malignancies are those selected from the group consisting of acute lymphoblastic leukemia, acute myelogenous leukemia, chronic lymphocytic leukemia, chronic myelogenous leukemia, Hodgkin's lymphoma, non-Hodgkin's lymphoma, and myeloma.

The cancer cells can be identified by known cell surface markers, for example, a cell surface marker for a solid tumor. The solid tumor can be a tumor of the breast, lung, colorectal system, stomach, prostate, ovary, uterus, cervix, kidney, pancreas, liver, brain, head and neck, nasopharyngeal system, or esophagus. In another specific embodiment, the cancer cell comprises a cell surface marker for a sarcoma. The sarcoma can be leiomyosarcoma, fibrosarcoma, rhabdomyosarcomas, or Ewing's sarcoma. In another specific embodiment, the cancer cell comprises a cell surface marker for a hematological tumor. The cancer cells can express one or cellular markers selected from markers such as CD3, CD10, CD19, CD20, CD22, CD23, CD25, CD30, CD33, CD35, CD37, CD38, CD40, CD44, CD52, CD70, CD80, CD133, CD200, epidermal growth factor receptor 1 (EGFR), epidermal growth factor receptor 2 (Her2/neu), human milk fat globule 1 (HMFG1), interleukin 2 receptor (IL2R), mucin 1, vascular endothelial growth factors, and the like.

In some embodiments, the disclosed methods and compositions can be applied to address autoimmune disorders, which are generally known and recognized in the art. Autoimmune conditions are disorders that arise from the dysfunction of the subject's immune system, wherein the immune system attacks and possibly destroys otherwise healthy body cells or tissue. In some embodiments, the disclosed methods and compositions can be applied to address autoimmune conditions that are associated with specific MHC gene sequences, such as specific HLA-I or HLA-II genes. Association of various autoimmune diseases with HLA genes are known. See, e.g., Thorsby and Lie, “HLA associated genetic predisposition to autoimmune diseases: Genes involved and possible mechanisms,” Transplant Immunology, 14:175-182 (2005) and Gough and Simmonds, “The HLA region and autoimmune disease: Associations and mechanisms of action,” Curr Genomics, 8:453-465 (2007), incorporated herein by reference. illustrative and non-limiting examples of relevant disorders that are autoimmune conditions associated, at least in part, with HLA sequences include: actinic prurigo, Addison's disease, ankylosing spondylitis, Behcet's, celiac disease, colitis, Crohn's disease, diabetes, drug hypersensitivity, Graves disease, haemochromatosis, Hashimoto's Thyroiditis, insulin-dependent diabetes mellitus, juvenile idiopathic arthritis, multiple sclerosis, myasthenia gravis, narcolepsy, osteoarthritis, polyarthralgia, polyarthritis, psoriasis vulgaris, reactive arthritis, rheumatoid arthritis, scleroderma, Sjogrens disease, systemic lupus erythematosus, and uveitis. In some embodiments, the disclosed methods and compositions can be applied to address autoimmune conditions that are associated with HLA-I gene sequences. Of interest are disorders associated with minor HLA antigens, such as occurs in some individuals post-transplant.

The compositions and methods of the present disclosure can be applied to immune cells in vivo (via administration to the subject), in vitro (to cells maintained in culture), or ex vivo (to immune cells isolated from a subject but maintained outside the subject). Ex vivo procedures, also referred to as adoptive immunotherapy is described in WO 20125/129514, incorporated herein by reference in its entirety. As described, the cells can be isolated from a subject by known techniques. The isolated population can be further enriched or depleted by known techniques, for instance affinity binding to antibodies and/or immunomagnetic selection. The cells can be expanded according to known techniques (see, e.g., U.S. Pat. No. 6,040,177, incorporated herein by reference). In any event, the cells can be a mixed population of cells, or enriched for a preferred population of cells as desired. In some embodiments, an exposure step is performed wherein the isolated cells are exposed to a synthetic polypeptide vaccine as described herein. In some embodiments, the cells are stimulated to express HLA-F, which promotes the HLA-F/MHC-I open conformer interaction on the cell surface. The synthetic polypeptide of the vaccine binds to the MHC-I conformer via the carrier epitope. The bound synthetic polypeptide is subsequently internalized and processed whereby the effector epitope/peptide is loaded onto an endogenous MHC-I or MHC-II molecule. The effector epitope/MHC complex is localized to the cell surface, whereby during a cultivation step the cell displays the effector epitope/MHC complex to an immune cell such as a T lymphocyte which is capable of stimulation upon binding of a T-cell receptor (TCR) to the effector epitope/MHC complex. The lymphocytes, for example, can be present in the initially isolated cell population or can be added subsequently. The stimulated lymphocytes can be re-introduced to the subject to realize the desired therapeutic or prophylactic effect. The procedure can be repeated over a course of therapy for a desired duration of several months or even years as may be necessary as may be determined by the attending health professionals.

The present synthetic polypeptides, fusion polypeptide, conjugates, nucleic acids, expression vectors, host cells, and antibodies, can be isolated and/or purified. The term “isolated” as used herein means having been removed from its natural environment. The term “puffed” as used herein means having been increased in purity, Wherein “purity” is a relative term, and not to be necessarily construed as absolute purity. For example, the purity can be at least 50%, can be greater than 60%, 70% or 80%, or can be 100%.

The present inventive synthetic immunomodulatory polypeptides, conjugates, nucleic acids, expression vectors, host cells, and antibodies (including antigen binding portions thereof), can be formulated into a composition, such as a pharmaceutical composition. In this regard, the present invention provides a pharmaceutical composition comprising any of the immunomodulatory polypeptides, fusion proteins, conjugates, nucleic acids, expression vectors, host cells, and antibodies, and a pharmaceutically acceptable carrier. The present pharmaceutical compositions can comprise more than one synthetic polypeptide or immunogenic material, e. g., a synthetic polypeptide and a nucleic acid, or two or more different polypeptides. Alternatively, the pharmaceutical composition can comprise an immunogenic material in combination with another pharmaceutically active agent or drug, such as a chemotherapeutic agents e.g., asparaginase, busulfan, carboplatin, cisplatin, daunorubicin, doxorubicin, uorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used and is limited only by chemico-physical considerations, such as solubility and lack of reactivity With the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use. The choice of carrier will be determined in part by the particular immunogenic material, as well as by the particular method used to administer the immunogenic material. Accordingly, there are a variety of suitable formulations of the pharmaceutical composition of the present invention.

The present invention also provides a vaccine comprising any of the synthetic polypeptides, fusion proteins, nucleic acids, expression vectors, and host cells described herein. The term “vaccine” as used herein means any substance as defined herein that causes modulation, such as activation, of an individual's immune system without causing actual disease. If the individual to be immunized is already afflicted with the disease, the vaccine can be administered in conjunction with other therapeutic treatments. Examples of other therapeutic treatments includes, but are not limited to, adoptive T cell immunotherapy, coadministration of cytokines or other therapeutic drugs for the disease.

The vaccine may be used either prophylactically or therapeutically. When provided prophylactically, the vaccine is provided in advance of any evidence of disease, e.g., cancer. The prophylactic administration of the vaccine should serve to prevent or attenuate the disease in an individual. In a preferred embodiment, individuals at high risk for the disease are prophylactically treated with the vaccines of the present invention. Examples of such include individuals with a family history of the disease or humans previously afflicted with the disease and therefore at risk for re-occurrence. When provided therapeutically, the vaccine is provided to enhance the patient's own immune response to the antigen, e. g., tumor antigen, present in the patient. In a preferred embodiment, the method provides for the killing of multiple tumor cells in a manner effective to treat cancer in the individual.

### EXAMPLES

The following examples merely illustrate aspects now contemplated for practicing the invention, but should not be construed to limit the invention. Provisional application 61/805,906, filed Mar. 27, 2013, is expressly incorporated herein by reference in its entirety. All literature citations described herein are expressly incorporated by reference for the specific purposes on which they are relied.

### Example I

In the present invention HLA-F and MHC-I interactions are used as mediators in the presentation of exogenous antigen by MHC class I. Viral, tumor, and minor histocompatibility antigens were examined for their capacity to stimulate class I restricted cellular responses when presented by HLA-F positive, MHC-I open conformer positive cells. The involvement of MHC-I open conformer and HLA-F interaction in this pathway was tested through direct binding of antigen and specific interference with their surface expression and transport. Mutant cell lines that lack a functional endogenous pathway and drugs that interfere with intracellular trafficking and protein degradation were used to distinguish the pathway from endogenous MHC-I presentation. These data collectively support a general mode of exogenous MHC-I antigen uptake and presentation by activated lymphocytes and monocytes that differs in significant detail from the presentation of endogenous antigen, providing a role for cooperation between HLA-F and MHC-I open conformers in this pathway.

Experimental Procedures

Cell Lines and Cultures.

NKL and KMA were all obtained from America Type Culture Collection (ATCC; Manassas, Va.) and cultured according to the product information sheet provided. B-LCL cell lines were previously collected and analyzed by the International Histocompatibility Workshops & Conference and obtained directly from the International Histocompatibility Working Group (IHWG) in Seattle (Hansen, 2006 Immunobiol Human MHC II). LCL 721.221 was obtained from the ATCC and maintained in RPMI 1640 medium supplemented with 10% v/v FCS, 2 mM L-glutamine and 1 mM sodium pyruvate. The related B-LCL 721 derivative TAP and Tapasin mutant cells, 0.134, 0.134C2 (TAP restored), 0.174, and T2 (hemizygous chr 6 derived from 0.174) were obtained from T. Spies (Fred Hutchinson Cancer Research Center, Seattle, Wash.). Other B-LCL cell lines were grown in RPMI medium supplemented with 10% FBS, 100 U/ml penicillin and 100 U/ml streptomycin. Genotyping for the presence of the HA-1H and R alleles was carried out as described (Tseng et al., 1998 Tissue Antigens 52: 305-311). The CTL clones specific for HIV, CMV and minor H antigens were derived in prior studies (Brodie et al., 1999 Nat Med 5: 34-41; Manley et al., 2004 Blood 104: 1075-1082; Tykodi et al., 2004 Clin Cancer Res 10: 7799-7811; each of which is incorporated herein by reference) and CTL clones specific for tumor antigen gp100 were also isolated previously (Yee et al., 1999 J Immunol 162: 2227-2234). CTL clones were expanded using the Rapid Expansion Protocol (Riddell and Greenberg, 1990 J Immunol Meth 128: 189-201). Typically 1-2×10⁵ freshly thawed CTL clone cells were added to 25×10⁶ irradiated PBMC and 5×10⁶ irradiated TM LCL in 25 ml CTL medium (RPMI-HEPES +10% Human AB serum and 55 μM 2-ME) with 30 ng/ml anti-CD3 antibody. After 24 hrs 50 U/ml rIL-2 (R&D Systems, Minneapolis, Minn.) was added and at 2 days the cells were washed and resuspended in CTL medium with 50 U/ml rIL-2. At days 7-8, and every 2-3 days thereafter, the cells were fed by replacing 50% of media with new CTL media plus 50 U/ml rIL-2. Cells were typically assayed between days 10 and 14.

Antibodies and Reagents.

mAbs 3D11, 4A11, 4B4, and 6A4 specific for HLA-F were generated as previously described (Goodridge et al., 2010 supra; Lee and Geraghty, 2003 J Immunol 171, 5264-5271; Lee et al., 2010, supra, each incorporated herein by reference). HCA2 was obtained from T. Spies, (Fred Hutchinson Cancer Research Center). Other mAb were purchased from suppliers including MA2.1 (ATCC, Manassas, Va.), Rab 5 (abcam, Cambridge, Mass.), Rab 7 (Cell Signal, Danvers, Mass.), CMV pp65 Tegument protein (UL83) mAb (Fitzgerald Industries Intl, Acton, Mass.). Proteins were obtained from the following sources: recombinant HIV-p24 (24 kDa, Prospecs, Ness-ziona, Israel) and recombinant pp65 protein (65 kDa, Miltenyi, Auburn, Calif.). Un-biotinylated and biotinylated 50 amino acid proteins were synthesized (Biosynthesis, Lewisville Tex.) as follows:

gp100#1,
[SEQ ID NO: 2]
YVWKTWGQYWQVLGGPVSGLSIGTGRAMLGTHTMEVTVYH RRG
SRSYVPL;
gp100#2, Bio-
[SEQ ID NO: 3]
SSGTLISRALVVTHTYLEPGPVTAQVVLQAAIPLTSCGSS
PVP GTTDGHR;
HA-1H, Bio-
[SEQ ID NO: 4]
DISHLLADVARFAEGLEKLKECVLHDDLLEARRPRAHECL
GEALRVMHQII;
HA-1R,
[SEQ ID NO: 5]
DISHLLADVARFAEGLEKLKECVLRDDLLEARRPRAHECL
GEALRVMHQII;
P17, Bio-
[SEQ ID NO: 6]
ASVLSGGKLDRWEKIRLRPGGKKKYKLKHIVWASRELERF
AVNPGLLETS;
P24, Bio-
[SEQ ID NO: 7]
IGWMTNNPPIPVGEIYKRWIILGLNKIVRMYSPTSILDIR
QGPKEPERDY.

Peptides were synthesized (Anaspec, Fremont, Calif.) as follows: pp65, HLA-A*0201, [SEQ ID NO:8] NLVPMVATV; gp100#1, HLAA*0201 [SEQ ID NO:9] KTWGQYWQV; gp100#2(g280), HLA-A*0201, [SEQ ID NO:10] YLEPGPVTA-K; HA-1H, HLA-A*0201 [SEQ ID NO:11] VLHDDLLEA; HIV-gag (p17), HLA-A*0301, [SEQ ID NO:12] RLRPGGKKK; HIV-gag (p24), HLA-B*2705, [SEQ ID NO:13] KRWIILGLNK.

Mass Spectrometry.

LC-MS/MS analysis of peptides and proteins was carried out at the Proteomics Shared Resource at the Fred Hutchinson Cancer Research Center, essentially as described previously (Goodridge et al., 2010, supra).

Immunostaining and Confocal Microscopy.

B-LCL cells (BM9) were incubated with 100 μg/ml biotinylated gp100#2 or HA-1H in RPMI+2% BSA for 4 hours at 37 C. After incubation, cells were washed and surface stained with pan class I mAb W6/32 labeled with alexa-488 (Biolegend, San Diego, Calif.). The cells were incubated for another 60 min with anti HLA-F mAb 3D11 or MHC-I HC mAb HCA2. After incubation, cells were washed and surface stained with W6/32 labeled with alexa-488 (Biolegend, San Diego, Calif.). The cells were then washed and intracellular staining performed with anti-IgG1-Dylight 649 (Jackson, West Grove, Pa.) for detection of anti-HLA-F and streptavidin alexa-594 (Invitrogen, Eugene, Oreg.) for detection of intracellular biotinylated antigens as per instructions (Intraprep, Beckman Coulter, Brea, Calif.). After intracellular staining, the cells were fixed with 1% paraformaldehyde, washed, resuspended in Prolong Gold antifade reagent with DAPI (Invitrogen, Eugene, Oreg.), and mounted onto optical slides. The cells were imaged using a Deltavision RT Wide-Field Deconvolution microscope (Applied Precision Inc, Issaquah, Wash.) and the images analyzed with ImageJ.

Cellular Assays.

B-LCL cell lines were pre-labeled with 50 μCi 51Cr for 1 h at 37 C and washed and incubated in the absence or presence of 10 μg/ml of Brefeldin A (BfA) (Biolegend, San Diego Calif.), 100 μM N-ethylmaleimide (NEM, Sigma-Aldrich, St. Louis, Mo.), 200 μM Chloroquine (Sigma-Aldrich, St. Louis, Mo.), or 200 μM Leupeptin (Sigma-Aldrich, St. Louis, Mo.) for 1 hour prior to addition of antigen. 5×105 51Cr labeled B-LCL cells suspended in 500 μl RPMI+2% BSA were incubated with peptide, vaccinia virus-pp65, recombinant protein (p24, pp65), or synthesized recombinant 50 amino acid proteins derived from their respective parent sequences for 4 hours at 37 C. These experiments resulted in establishing a protocol for proteins that required denaturing the proteins prior to the sensitization of cells, similar to that previously described (Barabas et al., 2008, supra). Following incubation with antigen, cells were washed three times and resuspended in RPMI+10% FBS and plated out at 5×103 cells per well with effectors at the indicated ratios. At 4 hours 30 μl of supernatant was collected and applied to lumaplates, dried and counted using a TopCount scintillation counter (Perkin Elmer, San Jose Calif.).

Temperature shift experiments were performed in an essentially similar protocol with cells incubated at either 4 C or 37 C for 1 hour before labeling with control peptide, p24, or p17 at the indicated concentration for a further 2 hours at 4 C or 37 C. For the centricon experiments, antigens were prepared by adding a 2× concentration of antigen to cells at 106 per ml suspended in AIM-V serum free medium or RPMI+2% FBS (no differences were observed) for 2 hours at 37° C. After incubation, cells were spun down at 1300 rpm and the supernatant collected and spun through a 3000 MW cut off centricon device. The flowthrough was applied at 1:2 starting dilution to the 51Cr labeled targets, in parallel to 51Cr labeled targets pulsed with control peptide or protein at the indicated concentration through serial dilutions. After incubation, the cells treated and analyzed as above. In peptide competition experiments, B-LCL or NKL cells were pre-labeled with 51Cr for 1 h at 37 C prior to pulsing with antigen. After labeling the cells were incubated for 1 hour with 100 μM A*02 peptide (C1R, Anaspec, Fremont, Calif.) at 37 C before the addition of positive control peptide or recombinant proteins for a further 4 hours.

Intracellular cytokine staining (ICS) was performed by directly adding recombinant pp65 to CTL clone 1C7-31 or the HA-1H 50 amino acid protein to CTL clone GAS#9, each resuspended in RPMI+10% Human AB serum. The cells were incubated for 1 hour at 37° C., followed by the addition of 10 μg/ml BfA and further incubation for 4 hours at 37° C. After incubation, cells were fixed and stained for intracellular IFN-G as per instructions (Intraprep, Beckman Coulter Brea, Calif.).

shRNA Knockdown Constructs.

Lentiviral vector (Horn et al., 2004 Blood 103: 3710-3716; Kurre et al., 2004 Mol Ther 9: 914-922) was used to construct β2m and HLA-F-specific knockdowns using synthetic oligonucleotides cloned into the Xba I/EcoR V cloning sites. shRNA construct targeting β2m had targeting sequence [SEQ ID NO:14] 5′-CAGCAGAGAATGGAAAGTCAA-3′ with forward oligonucleotide [SEQ ID NO:15]5′-CTAGACAGCAGAGAATGGAAAGTCAA CTCGAGTTGACTTTCCATTCTCTGCTGTTTTTTGAT-3′ and reverse oligonucleotide [SEQ ID NO:16] 5 ATCAAAAAAAGCAGAGAATGGAAAGTCAACTCGAGTTGACTTTCCATTCTC TGCTGT-3′; the construct targeting HLA-F had targeting sequence [SEQ ID NO:17] 5′-TGGTCGCTGCTGTGATGTGGAGGAAGAAG, with forward oligonucleotide [SEQ ID NO:18] 5‘-CTAGATGGTCGCTGCTGTGATGTGGAGGAAGAAGTCAAGAGCTTCTTCCTC CACATCACAGCAGCGACCATTTTTTGAT-3’ and reverse oligonucleotide [SEQ ID NO:19] 5′-ATC AAAAAATGGTCGCTGCTGTGATGTGGAGGAAGAAGCTCTTGA CTTCTTCCTCCACATCACAGCAGCGACCAT-3′. Italicized font represents the loop. A termination sequence (TTTTTT) is located immediately downstream of the reverse complementary sequence to terminate the transcription by RNA pol III. Construct was cotransfected with gag-pol transfer and envelope helper plasmids into 293T cells by calcium phosphate. Viral particles were harvested at 16 and 40 hrs, filtered by 0.45 μm pore size, and concentrated 100-fold by PEG-it™ (SBI, Mountain View, Calif.). All transductions were performed over four consecutive days. At day one, 60×104 B-LCL cells were seeded in 24-well plates in 1.5 ml RPMI 1640 with 10% PBS, 20 μl of 100-fold concentrated virus supernatant were added in the presence of 8 μg/ml protamine sulfate, and the plate centrifuged at 2500 rpm at room temperature for 1 hour. One ml of culture medium was replaced with 1 ml of fresh medium containing 20 μl of concentrated virus and protamine sulfate and centrifuged once per day for the next 3 days. GFP positive cells were analyzed and sorted by flow cytometry.

MHC-I Complex Formation.

T2 cells were washed twice with RPMI 1640 medium plus 10% BSA and resuspended in RPMI 1640-10% BSA at a concentration of 50×10⁴ cells/ml and 200 μM each of leupeptin and chloroquine were added. After 1 hour, 30 μg/ml HCMV protein pp65 or 2 μM pp65 peptide ([SEQ ID NO:20] NLVPMVATV) in DMSO was added. Urea buffer containing 50 mM NaH2PO4, 300 mM NaCl, and 4M urea or DMSO was added to control reactions. After 16 hours, cells were incubated with 10 μg/ml BfA for 4 hours and stained with MHC-I mAbs by FACS.

Subcellular Fractionation.

KOSE cells were suspended in hypo-osmotic buffer containing 10 mM Hepes pH 7.5, 15 mM KCl, 1.5 mM MgAc and 1 mM DTT at a concentration of 35×10⁶ cells/ml. Cells were homogenized with 15 strokes in a Pyrex glass homogenizer and 1/10 (v/v) of hyper-osmotic buffer containing 10 mM Hepes pH 7.5, 700 mM KCl, 40 mM MgAc, and 1 mM DTT was added to the final homogenate. After centrifugation at 800×g for 10 mM at 4° C., the nuclear pellet was washed with 10 mM Hepes pH 7.5, 85 mM KCl, 5.5 mM MgAc, and 1 mM DTT and collected by centrifugation. Combined post-nuclear supernatants were centrifuged at 145,000×g at 4° C. for 25 mM and the resulting crude membrane pellet was resuspended in 1 ml homogenizer buffer containing 10 mM Tris pH 8.0, 0.25 M sucrose, and 1 mM EDTA.

A 50% (wt/vol) sucrose stock solution in 10 mM Tris pH 7.5, 1 mM EDTA was used to prepare a 50-10% sucrose gradient using 11 dilution steps of 1 ml each. The gradient was equilibrated overnight at 4° C. The membrane suspension prepared above was layered on top of the gradient and centrifuged overnight at 100,000×g at 4° C. in a Beckman ultracentrifuge equipped with a SW-41Ti rotor. One ml fractions were collected and analyzed for marker enzymes by enzyme activity assays. Marker proteins (Rab 5, Rab 7), pp65 protein, HLA-F, and class 1 heavy chain were analyzed by Western blotting. The activity of lysosomal enzyme β-hexaminidase was assayed fluorometrically using 4-methylumbelliferyl substrates (Apollo Scientific, UK) as described (Johnson et al., 1972 Meth Enzymol 28: 857-861). Plasma membrane 5′-nucleotidase activity was measured as described (Coligan et al., 1999 Curr Prot Cell Biol Supp. 2, 3.2.1-3.2.16). For Western blot analysis, a 60 μl aliquot of each fraction was reduced by 5× sample buffer and separated by 12% Tris-glycine gel (Invitrogen, Carlsbad, Calif.). Specific protein was detected by mAb as indicated and visualized with a chemiluminescence system (Roche Applied Science, Penzberg, Germany).

Results

HLA-F is Down Regulated Via Internalization in Response to Exogenous Peptide or Protein.

HLA-F has previously been shown to be down-modulated on the surface of B-LCLs upon addition of MHC-I mAb specific for heavy chain (HC) or open conformer (OC) (Goodridge et al., 2010 supra). Since heavy chain specific mAb bind within the cleft region of MHC-I, in the context of the present invention it is believed that such mAb binding either structurally mimics peptide binding and thus alters the structure of MHC-I HC to resemble complex resulting in dissociation and down-modulation of HLA-F, or causes cross-linking of HLA-F/MHC-I heterodimers triggering internalization. Because the postulated structure of MHC-I when associated with HLA-F is open and thus peptide receptive, the possibility was considered that long polypeptides (≧30aa) with internal MHC-I epitopes may also bind to open class-I MHC, and that the binding of long polypeptides containing multiple MHC epitopes would produce cross-linking in the same way as mAb and thus down modulation of HLA-F.

HLA-F levels were compared before and after the addition of denatured viral proteins HIV-1 p24 or HCMV pp65. Marked decreases in HLA-F levels were indeed observed in both cases (FIG. 1A). The observed down-modulation suggested that HLA-F was being internalized in response to the addition of exogenous protein potentially interacting with MHC-I, HLA-F, or a related complex or structure on the cell surface. Fluorescence microscopy was then used to visualize the fate of HLA-F, MHC-I, and exogenous protein directly. A synthesized 50 amino acid polypeptide (Biotinylated gp100#2) derived from melanoma antigen gp100 (Yee et al., 2000 J Exp Med 192: 1637-1644) and a synthesized 50 amino acid polypeptide (biotinylated HA-1H) derived from the minor histocompatibility antigen HA-1H (den Haan et al., 1998 Science 279: 1054-1057) were incubated with target HLA-F positive B-LCL cell lines and visualized by intracellular staining with streptavidin-PE. The biotinylated proteins co-localized with HLA-F or MHC-I both on the cell surface and within the cell, supporting the belief that the molecules are internalized together (FIG. 1A). The overlapping intracellular signals may also indicate that both molecules remain co-localized during the initial stages of processing.

Exogenous Antigen Internalizes into Early Endosomes and Processing is Sensitive to Inhibitors of Lysosomal Enzymes.

Given the co-localization of HLA-F, open MHC and antigen, it was next attempted to trace the passage of antigen from the extracellular space, through internalization, and back to the surface as MHC-I peptide complex. First, complex formation was examined on the surface of T2 cells in the presence or absence of added antigen using conformation specific mAbs mA2.1 (HLA-A*02 specific) and W6/32 (pan MHC complex specific). These experiments were designed to evaluate an increase in MHC complex formation after addition of antigen as a means of gauging cross-presentation of target antigen and are similar in design to a previously described assay that measured relative MHC-I peptide affinities (Abdul-Alim et al., 2010 J Immunol 184: 6514-6521). To optimize the concentrations of control nonamer peptide and target antigen to be used before and after drug treatment, titrations of peptide and protein were carried out in the absence of drugs (Fig. S1). The midpoint or half maximal concentration of antigen for both protein and peptide was used in triplicate experiments and the mean florescence index (MFI) of mAb binding before and after addition of peptide and protein was compared in the presence of two lysosomal inhibitors and Brefeldin A (BfA). While the change in MFI for control nonamer peptide was not significantly affected by any of the drugs, the increase in MFI for both mA2.1 and W6/32 observed after addition of exogenous pp65 protein was virtually eliminated by all three inhibitors tested (FIG. 1B).

Next, to observe the pathway and processing of antigen more directly, cells were fractionated after the addition of exogenous pp65 antigen and analyzed the proteins by western blot with pp65 specific mAb. Enzyme activity for each fraction was measured to identify the subcellular compartments contained within each fraction. The enzyme markers present in fractions 1-4 and 9-10 were consistent with the presence of early endosomes and lysosomes, respectively. These fractions also contained pp65 protein, which migrated at a reduced molecular weight (MW) in all fractions. The species of pp65 in the gradient fractions containing lysosomes had further reduced MW relative to the fractions containing early endosomes, suggesting additional processing of protein (FIG. 1C). These data, taken together with the drug sensitivity data, were consistent with antigen entering cells, passing through early endosomes, and proceeding through lysosomes before the derivative peptide was generated. Derivative peptide then combined with MHC-I and the resultant complex is expressed on the surface (as detected by W6/32 and mA2.1).

Binding of Surface MHC-I to Exogenous Protein is Dependent on MHC-I Specific Epitopes.

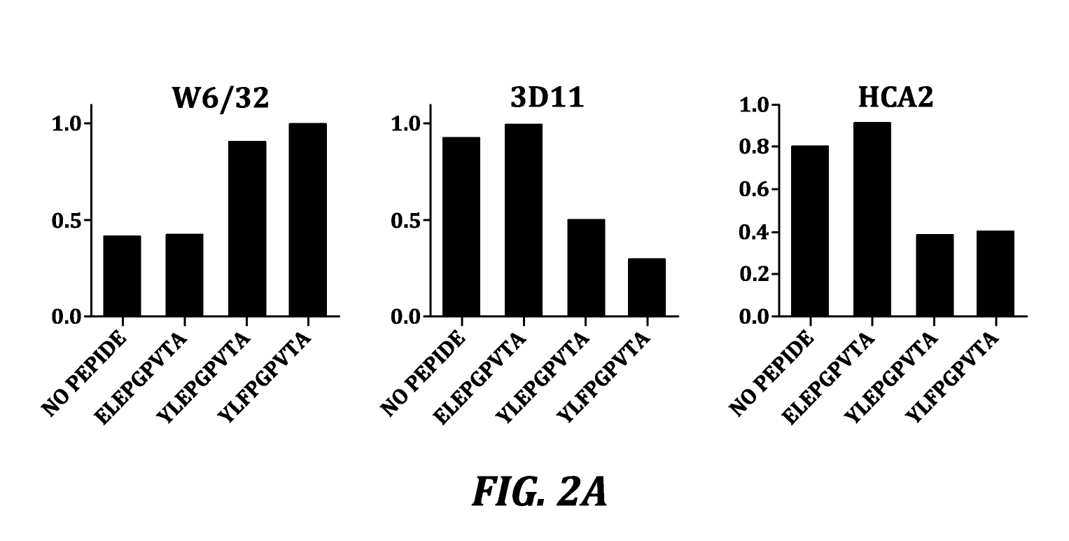

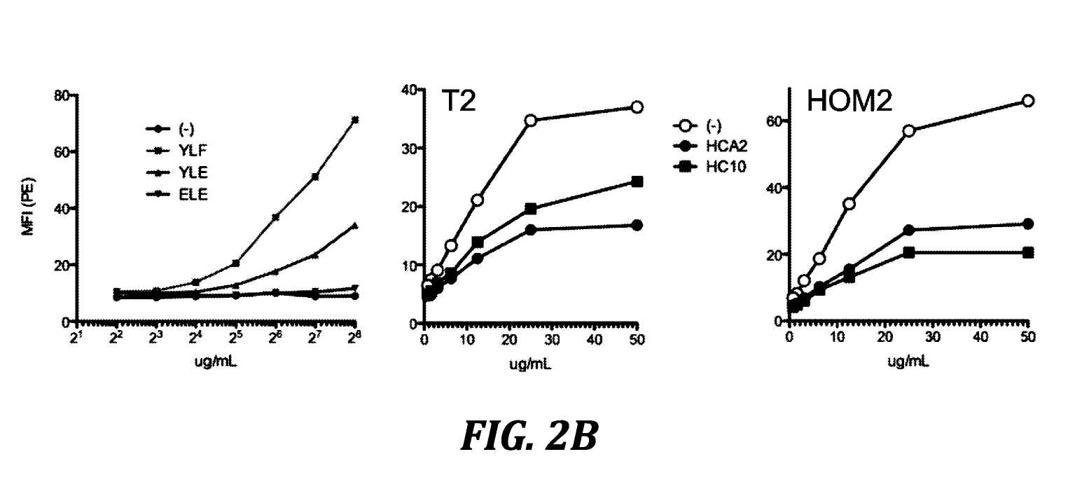

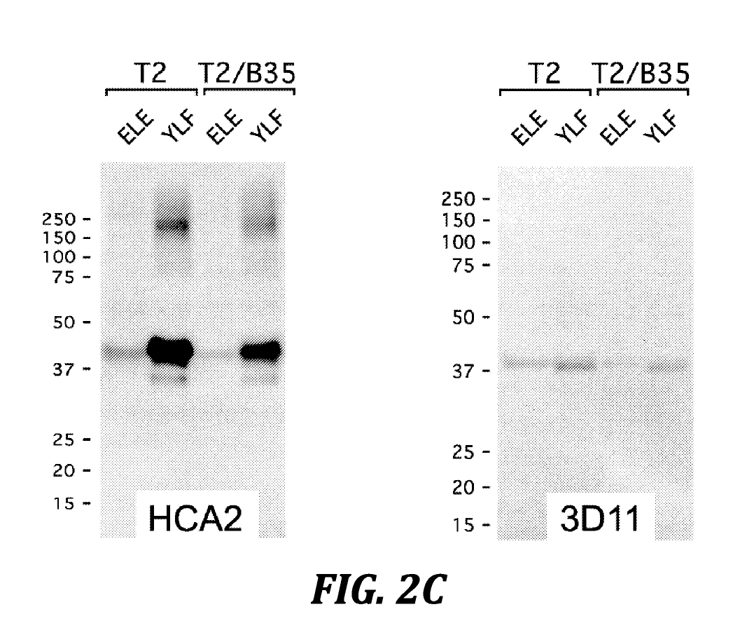

Given coincident internalization of exogenous antigen with MHC-I and HLA-F, whether direct physical interaction occurs between exogenous antigen and MHC-I in an epitope specific manner was examined. An established experimental system was used that identified HLA-A*0201 specific high affinity mutant peptides derived from tumor antigen gp100 (Parkhurst et al., 1996 J Immunol 157: 2539-2548). Three peptides were chosen from that system, differing from one another by successive single amino acid changes, that bound with low (ELE), medium (the naturally occurring sequence YLE), or high (YLF) affinities to HLAA*0201. These peptides were used to measure relative increases in MHC-I complex formation using the T2 system described above. The ability of the peptides to increase levels of MHC-I complex was in direct relation to their binding affinity for HLA-A*0201 as was a corresponding reduction in the levels of MHC-I open conformer (through complex formation) and HLA-F (FIG. 2A). These data suggested that higher affinity peptides spontaneously formed complexes with MHC-I more readily, resulting in increased levels of HLA-F/MHC-I OC dissociation and consequent reduced levels of surface HLA-F.

To test whether specific MHC epitopes contained within an extended polypeptide affected their ability to bind open conformers of MHC-I, three N-terminally biotinylated 50-amino acid protein fragments of gp100 were synthesized-one that included the native nonamer sequence and two derivatives that contained the low and high affinity mutant sequences. The relative binding of each polypeptide to T2cells reflected the binding affinity of the epitope sequence contained within it, suggesting a direct interaction of the extended polypeptide with open MHC-I, as seen with the corresponding nonameric peptides. In support of this interpretation, mAbs against MHC-I open conformers were able to block relative binding of the high affinity protein to either T2 cells or to B-LCL HOM2 (FIG. 2B). To examine the interaction of the peptide sequence with MHC-I and possibly HLA-F, the low and high affinity polypeptides were used in comparative precipitation experiments using T2 cells and T2-HLA-B*35 transfectants. Western blot analysis of precipitated material showed that the high affinity polypeptide consistently bound to quantitatively higher levels of both MHC-I and HLA-F, reflecting the surface binding abilities and relative affinities of the epitopes contained within the polypeptides (FIG. 2C).

Exogenous Protein Sensitizes HLA-F Positive Targets to CTL Recognition

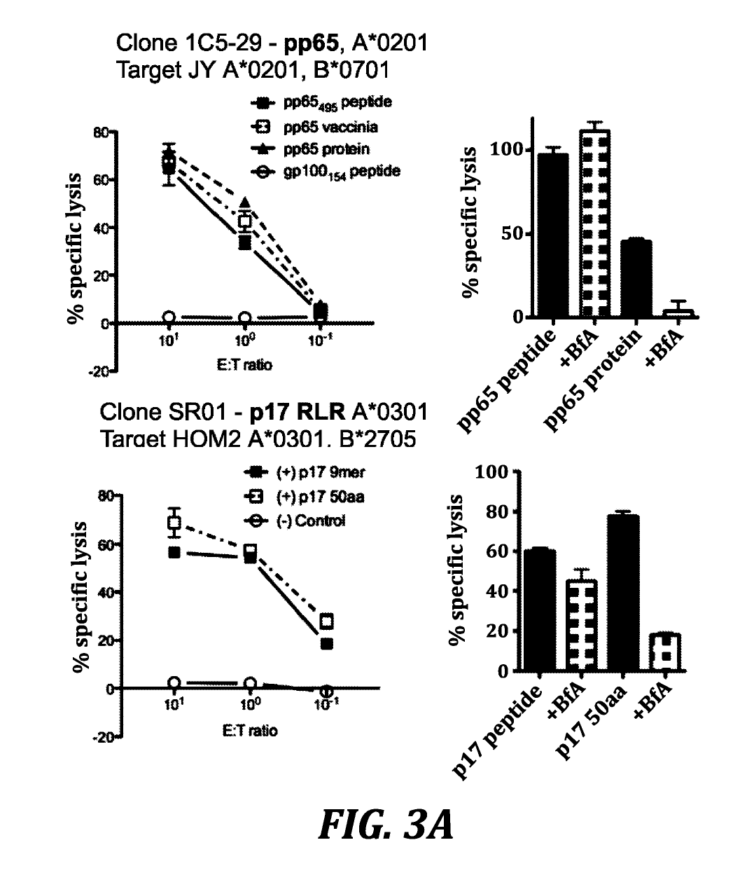

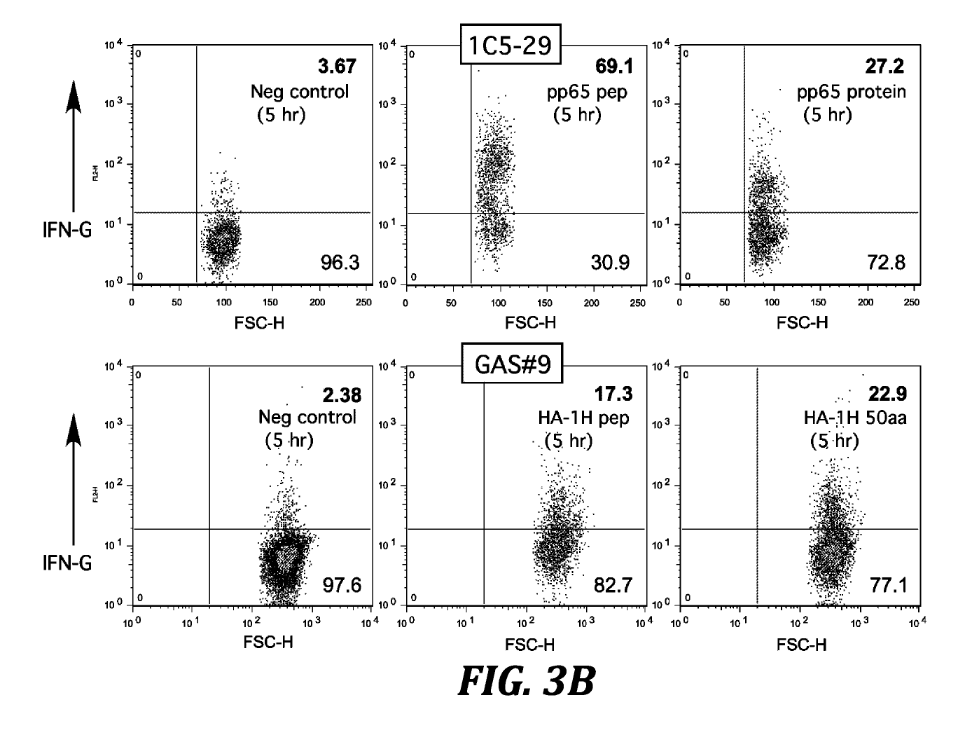

The coincident internalization of exogenous antigen with MHC-I and HLA-F combined with epitope specificity of MHC-I binding suggested a pathway for antigen presentation distinct from existing models for cross-presentation by MHC-I (Basta and Alatery, 2007, supra; Guermonprez and Amigorena, 2005 supra; Monu and Trombetta, 2007 Curr Opin Immunol 19: 66-72.; Shen and Rock, 2006 supra). To explore a role for the interaction of MHC-I open conformer and HLA-F in antigen uptake for processing and presentation, available CTL clones were used specific for epitopes HIV-gag p17 and CMV pp65 and tested whether BLCL target cells that express both HLA-F and the MHC-1 restricting allele could be specifically sensitized to lysis by CTL effectors upon the addition of exogenous antigen. For a number of different combinations of target cell, exogenous protein (HIV-gag p17 and CMV pp65), class I HLA restricting allele (HLA A*0201, A*0301, B*2705) and CTL, conditions were defined where exogenous proteins optimally sensitized B-LCL to lysis by specific CTL. BrefeldinA was used to distinguish between two possibilities that could confer sensitivity to CTL lysis: internalization and processing of exogenous protein or spontaneous formation of MHC-I complex by direct addition of peptide (FIG. 3A). BfA inhibits transport of proteins through the Golgi and induces retrograde protein transport from the Golgi to the ER, thus distinguishing presentation of internalized and processed protein from spontaneous complex formation by direct addition of peptide.

Although inhibition of lysis by BfA discriminated between exogenous protein and direct peptide addition, three additional control experiments were performed to exclude the possibilities that the protein preparations were contaminated with degenerate peptides or were degraded to release corresponding nonamer peptides during the course of the experiments. First, the presence of small amounts of contaminating peptide was tested by subjecting each protein preparation to LC-MS/MS and examining the spectra for the targeted specific peptides and extended peptides containing the specific peptide sequence (for up to 4 amino acids extending in both directions). No peptides containing the target epitopes were detectable in any of the preparations (Fig. S2A, B). Next, to examine the possibility that peptides might be spontaneously generated from proteins during incubation with target cells, a mock incubation was performed identical to experimental conditions used for sensitization followed by fractionation. Peptide titrations before or after centricon pass-through were overlapping, demonstrating effective recovery of pM concentrations of peptide. In contrast, the pass-through from long polypeptide preparations was ineffective for sensitization to lysis at all concentrations tested, down to the lowest concentrations of protein effective for sensitization (Fig. S2C).

The third control experiment was based on an observation that surface binding of biotinylated p17 and p24 antigen to B-LCL was reduced substantially at 4° C. versus 37° C. for both polypeptides (Fig. S3A). Thus, if antigen uptake and processing was required, sensitization to lysis would be impaired at 4° C. but not at. 37° C. Conversely, direct addition of peptide should not be impaired at 4° C. as peptide uptake is not required for complex formation (Barabas et al., 2008 supra). For both polypeptides, the ability of exogenous antigen to sensitize B-LCL to lysis was impaired at lower temperature while direct addition of peptide was unaffected (Fig. S3B). In combination with the blockade of presentation by BfA, these control experiments support the conclusion that specific peptide is neither present in the original protein preparations nor generated external to cells during the course of the experiments prior to exposure of targets to effectors.

The data support a pathway in which exogenous antigens are internalized, processed and presented by MHC-I by B-LCLs—one potentially involving HLA-F. However, surface expression of HLA-F is upregulated in most lymphocyte subsets upon activation, including activated T cell clones (Lee et al., 2010 supra). Thus, this pathway might also function in other cell types that express HLA-F. To test this in T cells, antigen specific, HLA-F positive CTL clones were pulsed with antigen directly in the absence of a target cell line and assayed for their ability to act as self-stimulators. The observed increase in IFN-G expression after exposure to extracellular antigen suggested that T cell clones apparently acquire, process and present exogenous antigen, suggesting that the effector cell itself can be involved in expansion of a memory response by cross-presenting antigen once stimulated (FIG. 3B). The ability of activated effectors to recruit antigen via this pathway suggests that antigen cross-presentation of fragmented target proteins may occur at the site of inflammation during effector responses.

HLA-F and MHC-I Open Conformer in Cross-Presentation

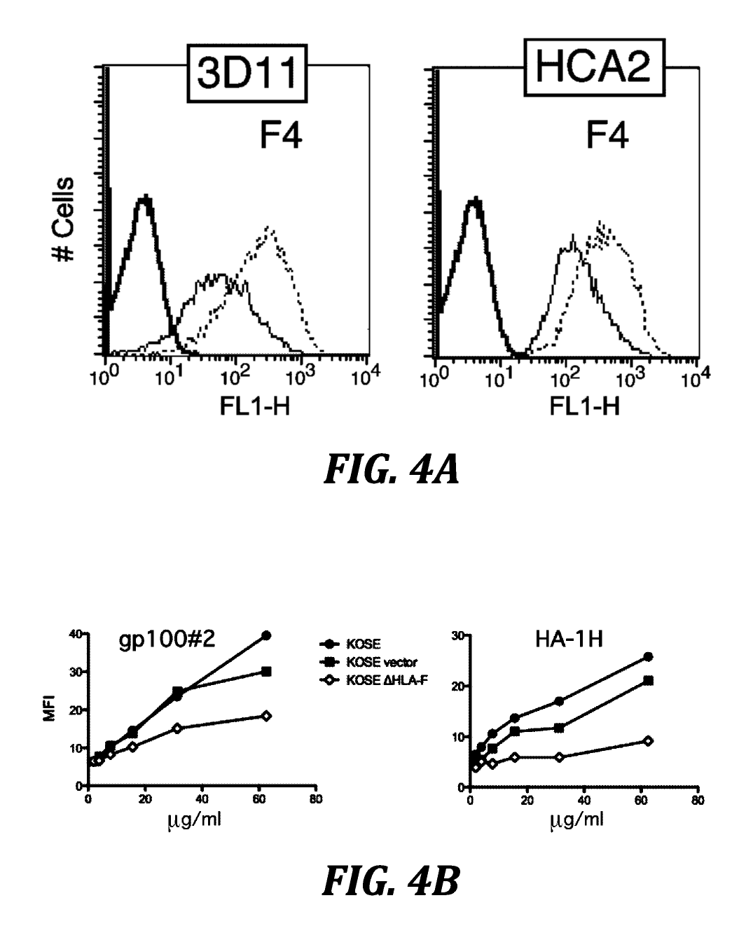

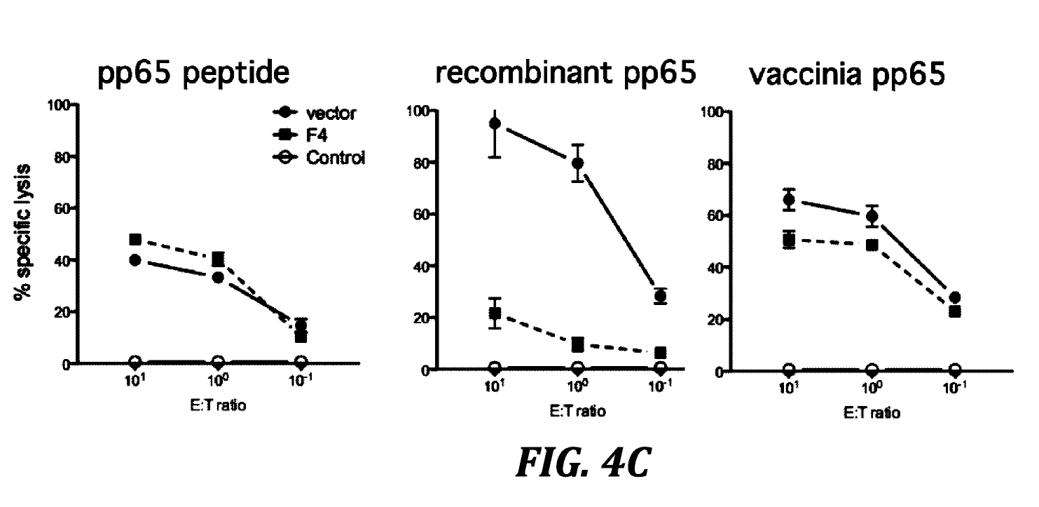

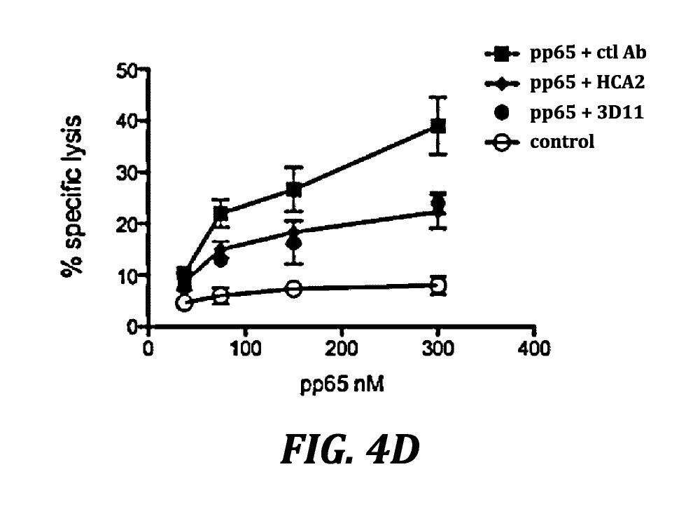

To examine the involvement of HLA-F in the novel pathway directly, shRNA knockdowns were designed targeting HLA-F, using lentivirus constructs to express the shRNA (Horn et al., 2004, supra). B-LCL KOSE cells expressing HLA-A*0201 were transfected with the construct and with control vector. Four distinct sequence constructs were screened for HLA-F and one selected that effected maximal down regulation of HLA-F. Surface levels of HLA-F were markedly decreased using the F4 shRNA construct, which also coincidentally reduced MHC-I heavy chain expression (FIG. 4A). The down regulation of MHC-I in the F4 transductants may be related to the intracellular interactions previously detected between HLA-F and MHC-I (Goodridge et al., 2010 supra).

Binding of biotinylated proteins to the surface of cells transduced with F4 was examined as an indirect measure of the effect that HLA-F and MHC-I HC levels might have on antigen binding. Both 50-amino acid polypeptides derived from gp100 and HA-1H showed marked reductions in surface binding on cells treated with HLA-F specific shRNA (FIG. 4B). Further, when tested for sensitization to lysis by a pp65 specific CTL clone, uptake and processing of exogenous pp65 protein was significantly impaired in F4 transfectants. This was in contrast to the recognition of target cells that occurred with endogenously synthesized pp65 after vaccinia/pp65 infection or those pulsed with specific peptide. In those cases both knockdowns compared similarly to vector only-transfected control (FIG. 4C). Sensitization assays incorporating mAb HCA2 or 3D11 blocking also demonstrated that specific interference of MHC-I open conformer or HLA-F prior to addition of exogenous protein affected antigen uptake and subsequent processing for MHC-I presentation (FIG. 4D).

Processing and Presentation of Exogenous Antigen for MHC-I is Independent of TAP and Tapasin

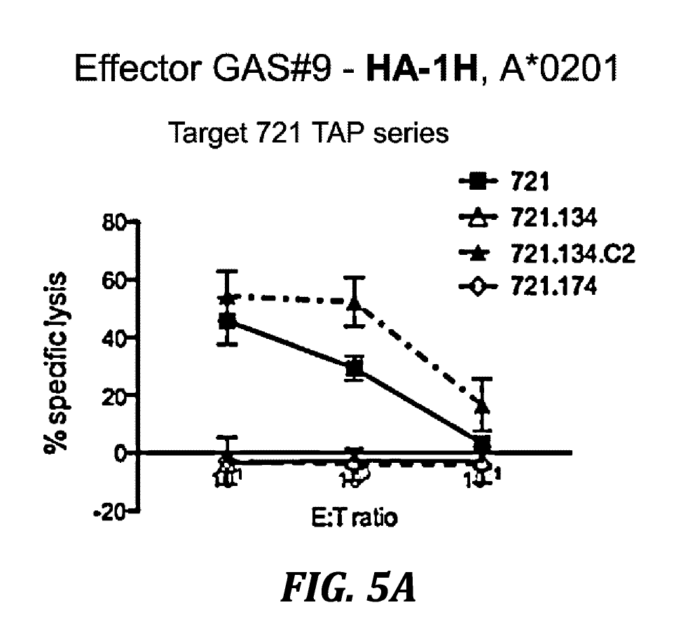

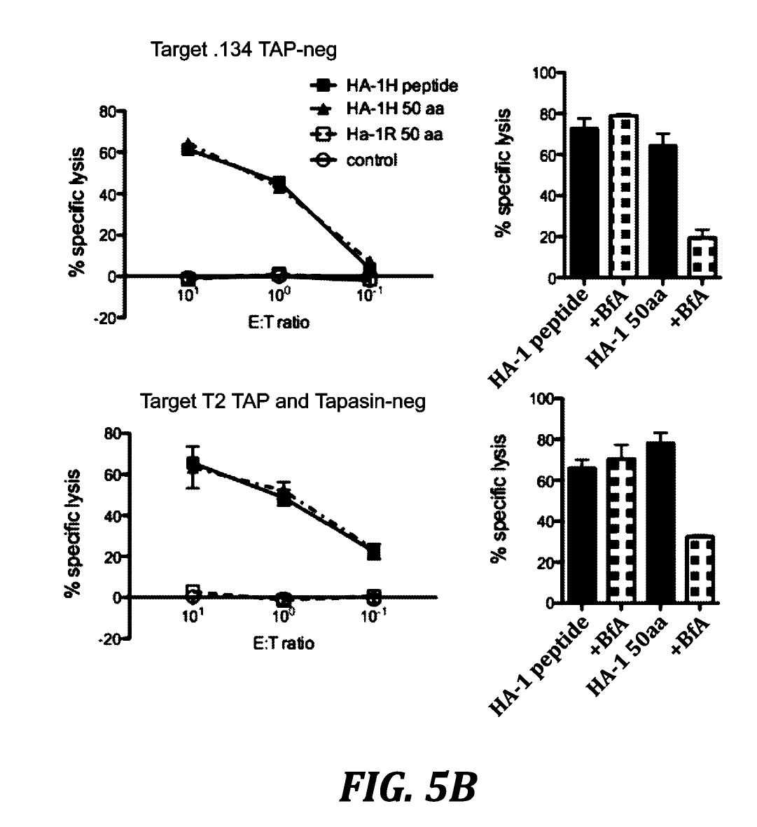

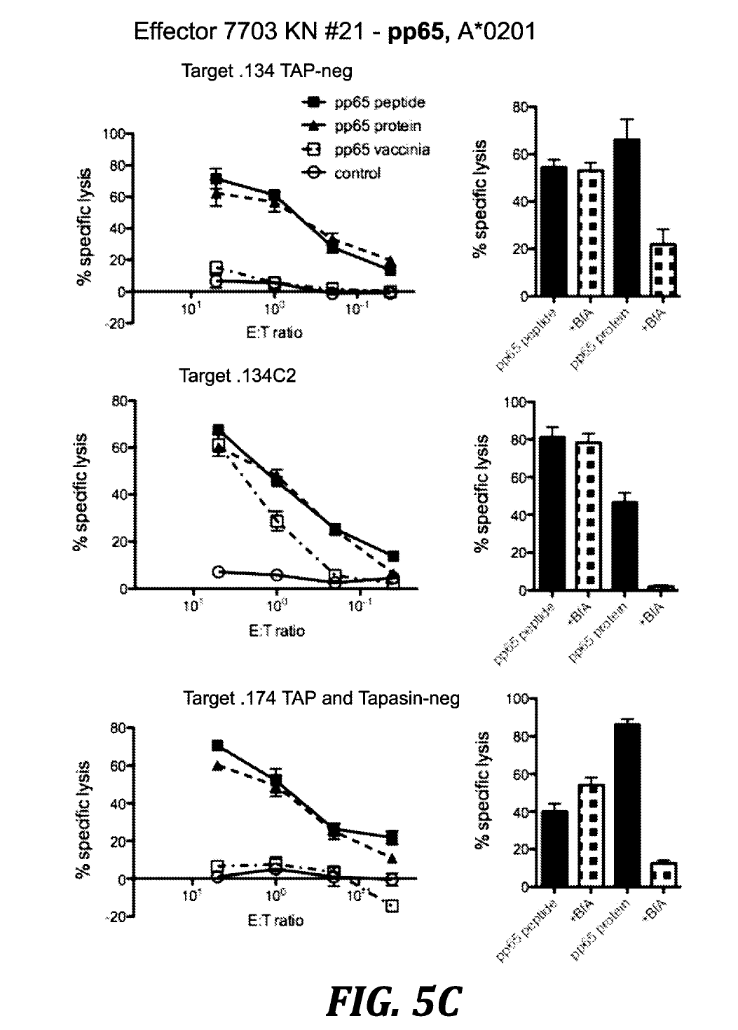

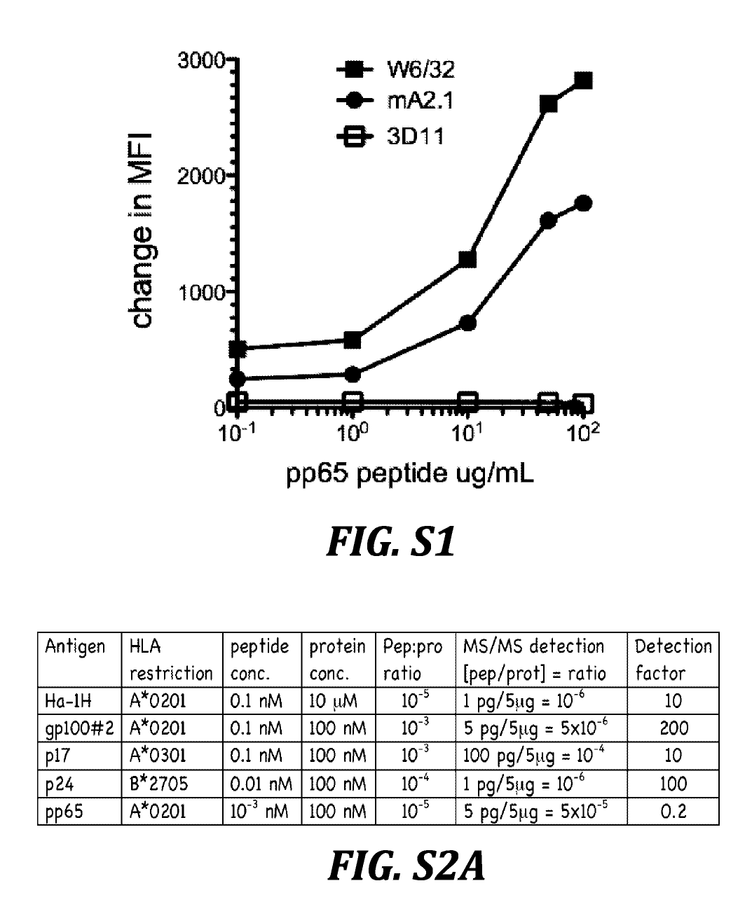

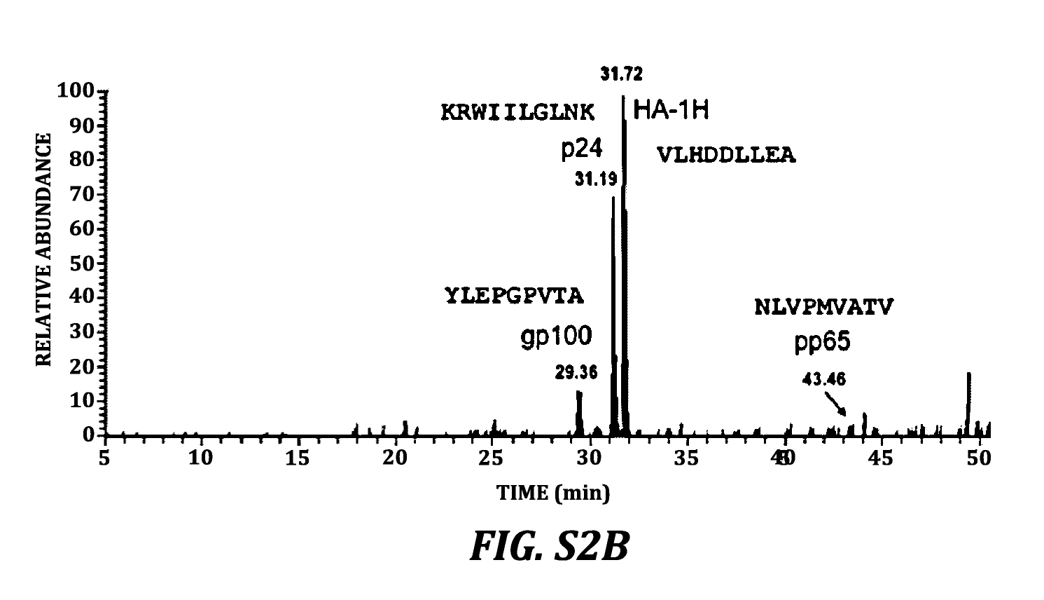

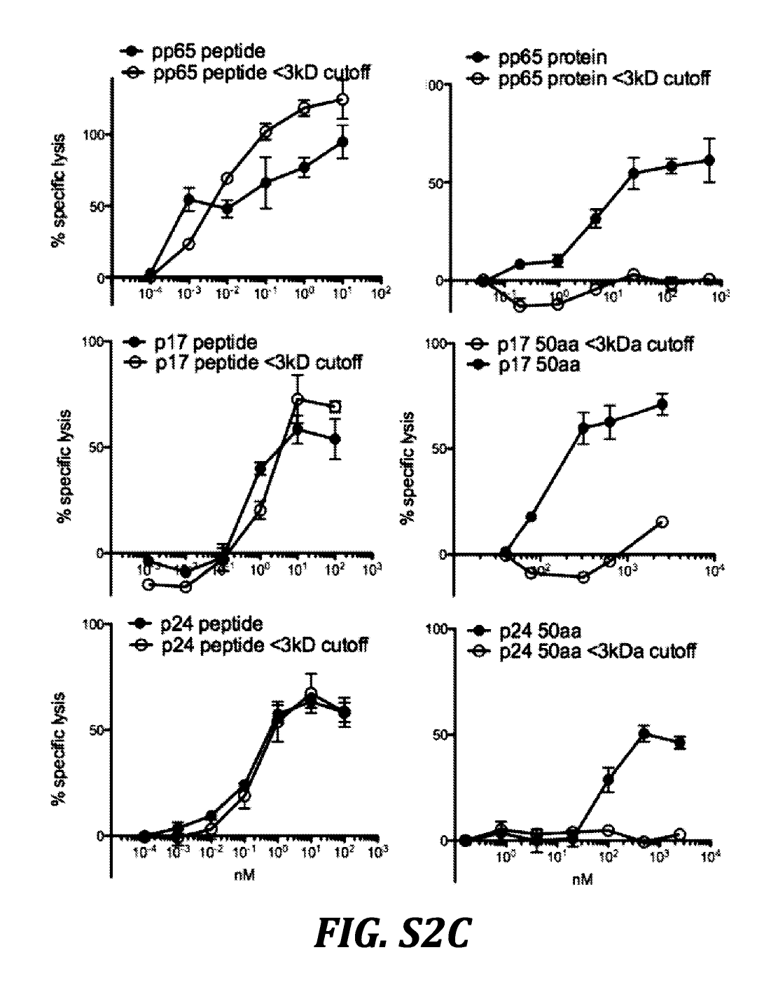

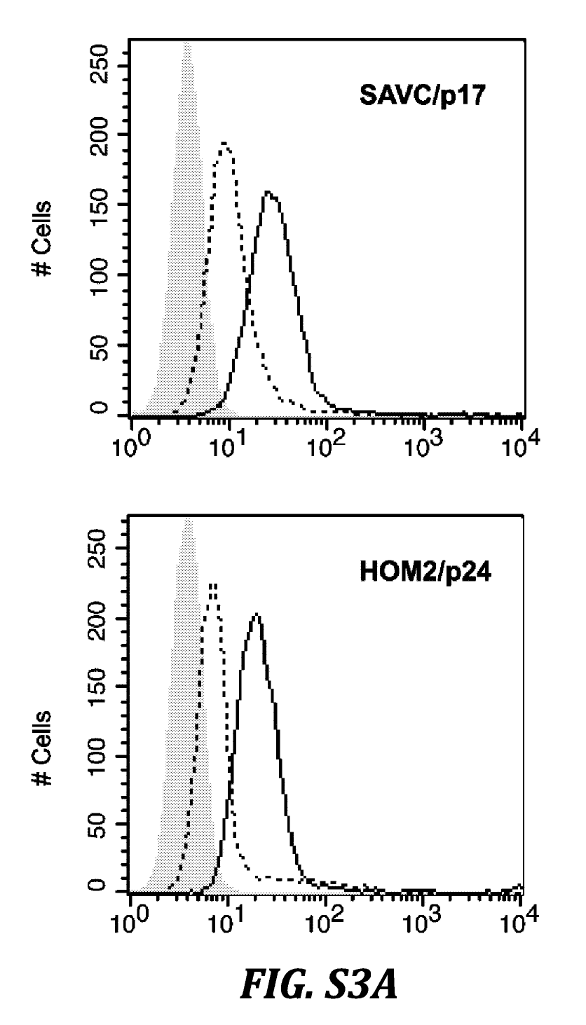

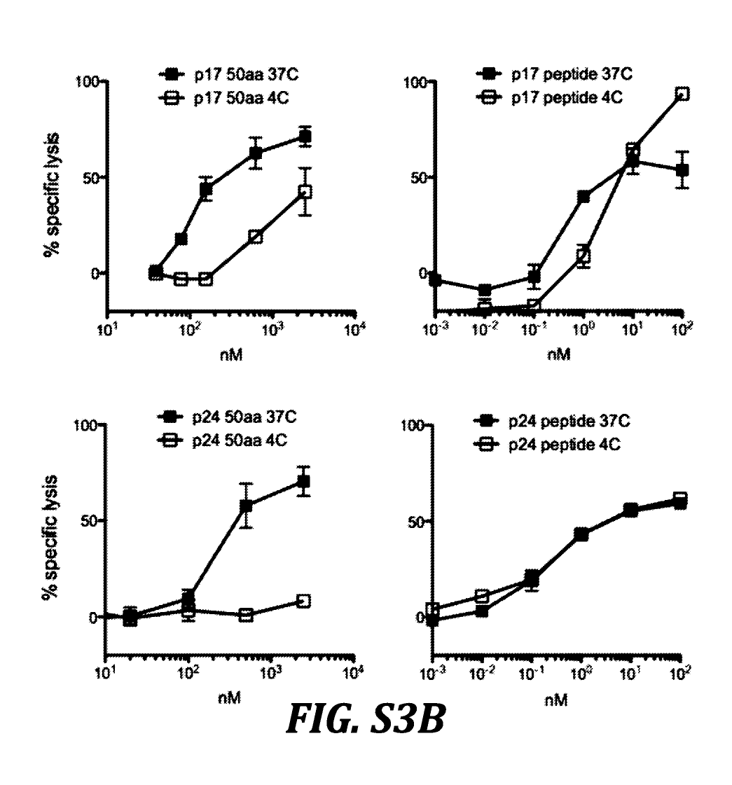

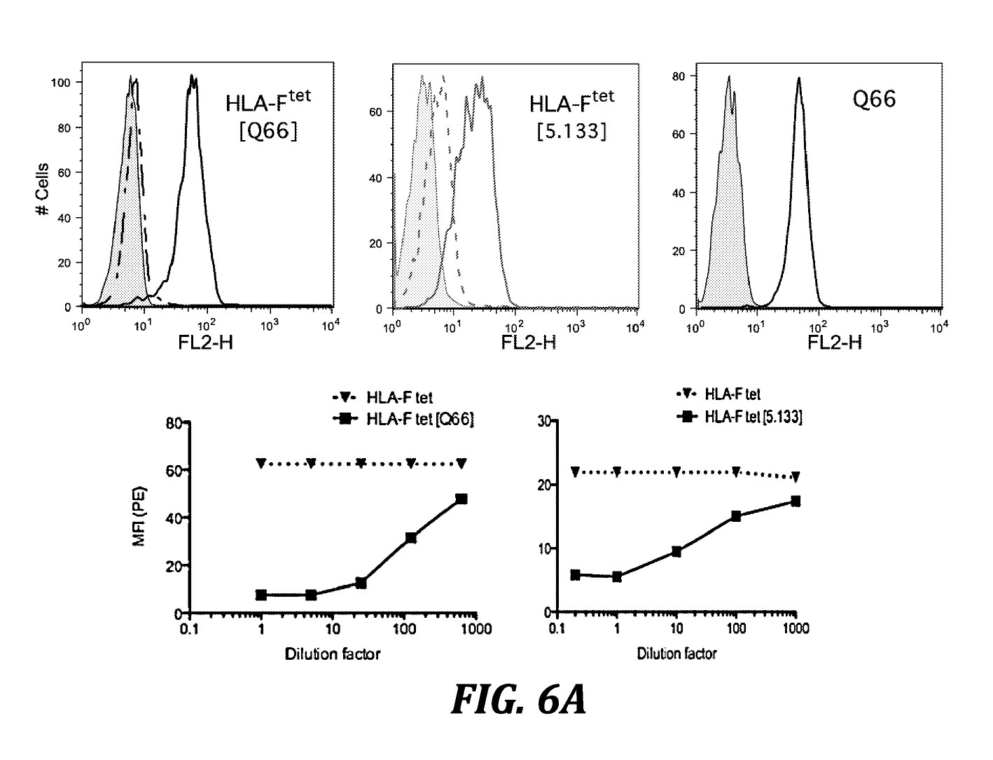

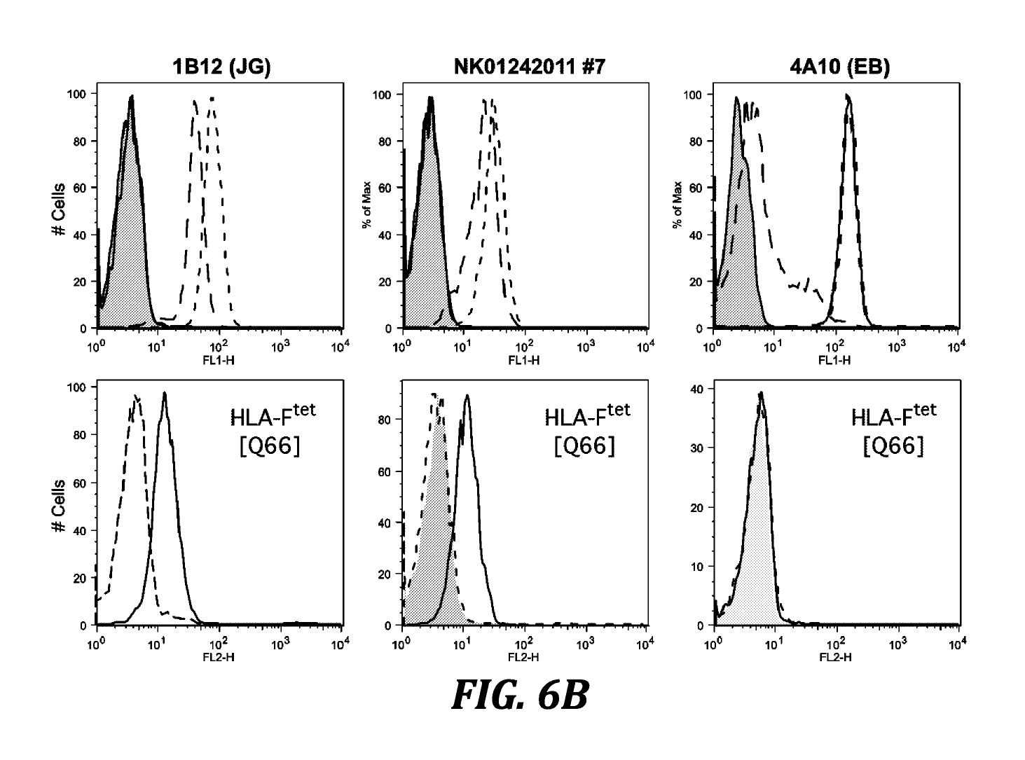

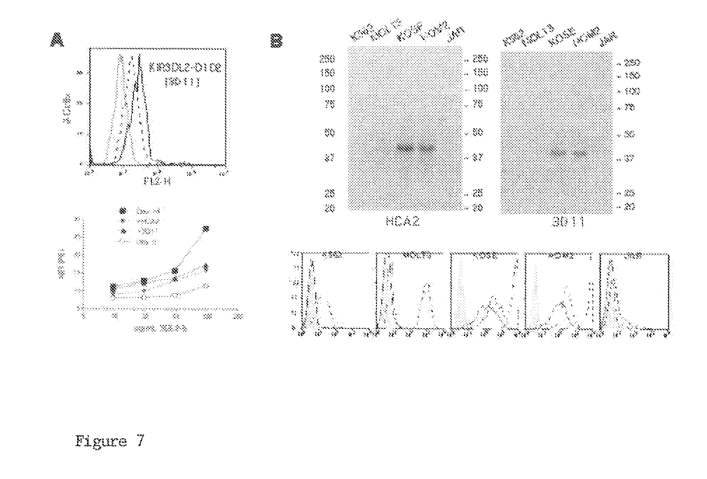

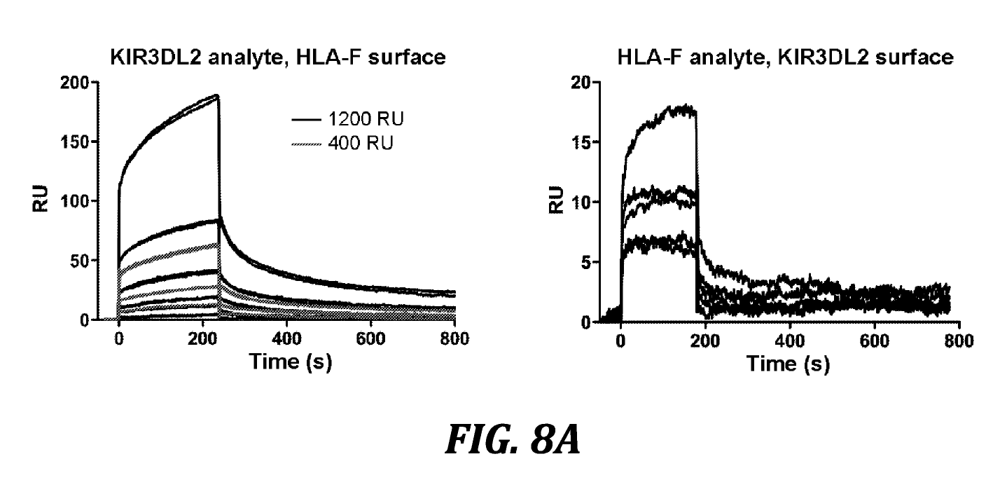

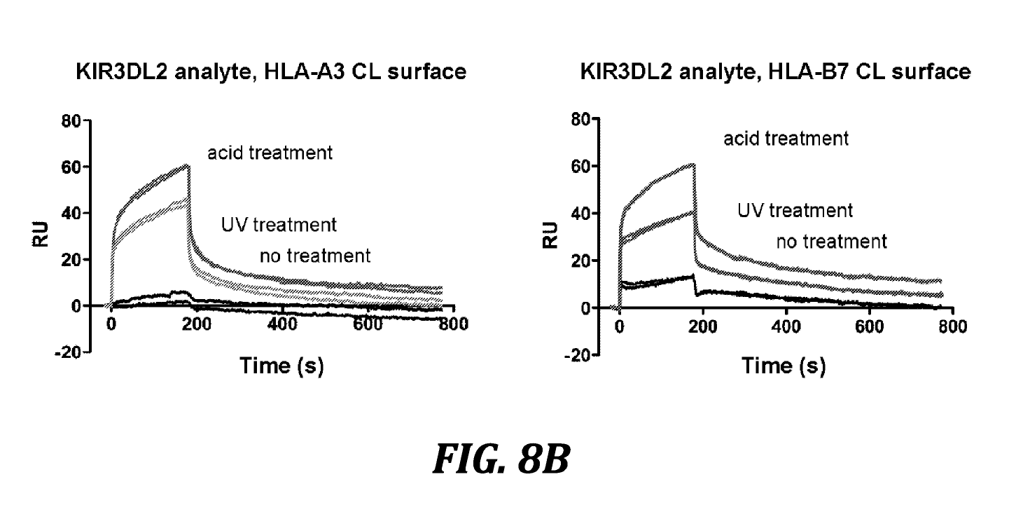

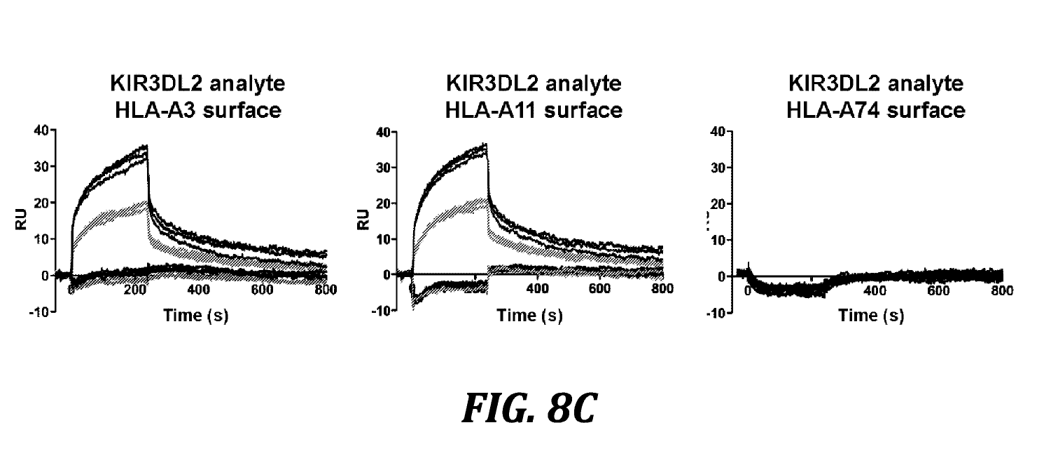

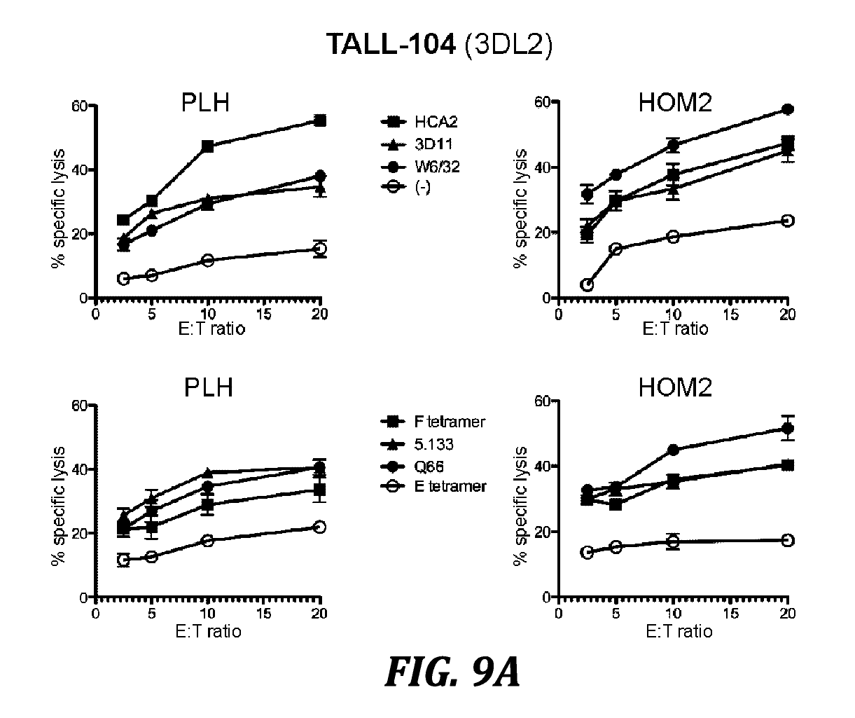

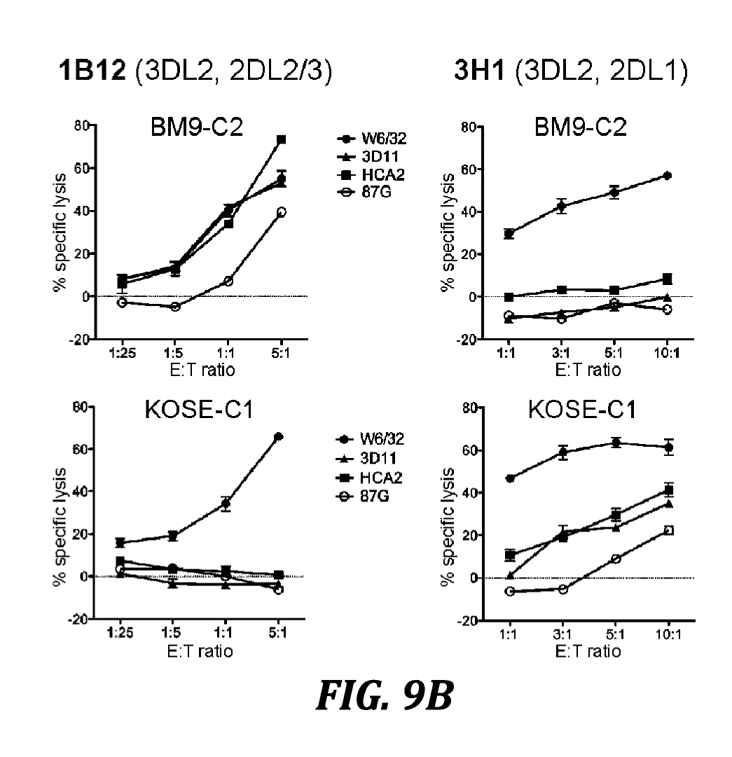

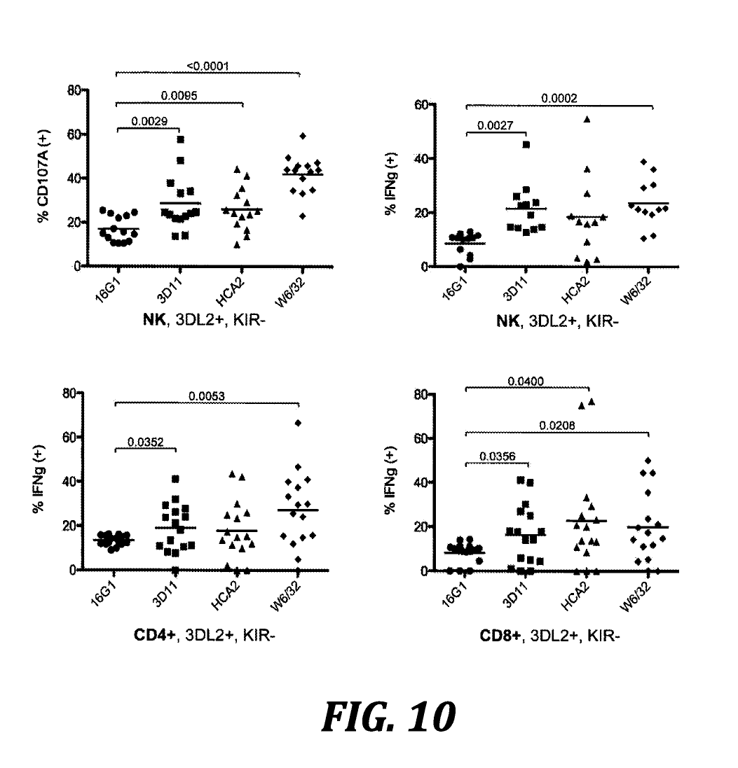

The endogenous class I antigen presentation pathway and its dependence on TAP and Tapasin is known (Raghavan et al., 2008 Trends Immunol 29: 436-443; Vyas et al., 2008 supra). To characterize exogenous MHC-I presentation, two antigen sources were examined where endogenous presentation and exogenous presentation could be compared in TAP and Tapasin mutant lines. It was first established that the HA-1H antigen was TAP dependent when presented through the endogenous pathway. B-LCL 721 was typed for HA-1 alleles using an established protocol (Tseng et al., 1998 supra) and found to be HA-1H homozygous. Dependence on TAP was confirmed when a panel of 721 and derivative.134 (TAP negative), 0.134C2 (TAP restored), and class II deletion mutant 0.174 (TAP and Tapasin deficient) were tested with HA-1H specific CTL. Only 721 and TAP restored 0.134C2 cells were sensitive to lysis by HA-1H-specific CTL (FIG. 5A). The same panel was next tested for access by exogenous antigen using the HA-1H 50-amino acid polypeptide. In contrast to endogenous antigen, HA-1H CTL lysed both 0.134 and 0.174 mutant lines pulsed with HA-1H protein and, consistent with prior experiments, BfA inhibited presentation of exogenous protein but not peptide (FIG. 5B).

Confirmation of the independence of this pathway from TAP and Tapasin was obtained with pp65-specific CTL where the presentation of endogenous pp65, expressed with a vaccinia virus construct, could be compared directly with exogenous protein using the same panel of LCL mutants. CTL specific for an HLA-A*0201 restricted pp65 epitope only recognized the TAP restored mutant 0.134C2 infected with vaccinia-pp65. This result is consistent with prior studies showing that endogenous pp65 antigen presentation is TAP and Tapasin dependent (Ortmann et al., 1997 Science 277: 1306-1309). In contrast, exogenous pp65 protein sensitized all targets including the mutant cell lines. For all targets, sensitization to lysis by protein was inhibited by BfA while peptide sensitization was not altered (FIG. 5C).

### Example II

In this Example, plasmacytoid dendritic cells (pDCs) were isolated from PBMC by first depletion of non-pDCs and then positive selection of pDCs using MicroBeads against the pDC-specific antigen CD304 (BDCA-4/Neuropilin-1) (Miltenyi Biotec #130-097-240). The isolated pDCs are CD303 (BDCA-2)+, CD304 (BDCA-4/Neuropilin-1)+, CD123+, CD4+, CD45RA+, CD141 (BDCA-3)dim, CD1c (BDCA-1)−, and CD2−. The cells lack expression of lineage markers (CD3, CD14, CD16, CD19, CD20, CD56) and express neither myeloid markers, such as CD13 and CD33, nor Fc receptors, such as CD16, CD64, or FcεRI. Binding of the antibody to CD304 (BDCA-4/Neuropilin-1) does not have a significant effect on IFN type I production in pDCs. For the depletion step, all non-pDCs were magnetically labeled by using cocktails containing specific MicroBeads, biotin-conjugated antibodies, and Anti-Biotin MicroBeads. For the subsequent positive selection step, the enriched pDCs were magnetically labeled with CD304 (BDCA-4/Neuropilin-1) MicroBeads.

Isolated pDCs were then cultured in RPMI1640-HEPES plus 10% human AB serum with 1) ODN2216 20 μg/ml, or 2) NIH3T3-tCD40L+IL3 20 ng/ml. Surface staining was done three days after culture. pDCs were stimulated with CD40L according to published procotols or by Class A CpG oligonucleotide—human TLR9 (Toll-like receptor 9) ligand. CpG ODNs are synthetic oligonucleotides that contain unmethylated CpG dinucleotides in particular sequence contexts (CpG motifs). These CpG motifs are present at a 20-fold greater frequency in bacterial DNA compared to mammalian DNA. CpG ODNs are recognized by Toll-like receptor 9 (TLR9) leading to strong immunostimulatory effects. Cells were stained with HLA-F specific mAbs and control mAb. Only the TRL9 ligand approach upregulated HLA-F, indicating the HLA-F pathway for antigen uptake functions in pDCs stimulated by specific protocols.

Thus, to stimulate a naïve immune response the professional antigen presenting cells targeted for uptake require specific stimulatory signals that upregulate HLA-F.

### Example III

To better define criteria for polypeptides to enter the HLA-F/MHC-I OC pathway, an in vitro system was developed for presentation by B cells to autologous PBMC. Frozen leukapheresed cells from HIV-1 nonprogressors were used as sources for both B cells as antigen targets and PBMC for stimulators to examine memory responses to HIV-1 antigens. As a demonstration that this system can detect specific responses three polypeptides were designed based on the HIV-1 p24 sequence, one with a wild type B*57 and two escape mutant epitopes, and all containing a B*27 and DRB1*01 epitope. This experiment demonstrated a robust expansion of HLA-B*27-KRW reactive T cells for all three polypeptides. The response against the HLA-B*57 epitope was more modest but did reflect the presence and absence of the requisite epitope as predicted from previous experiments, with only the native TST polypeptide evoking a response. In this demonstration of MHC-II specific expansion significant levels of HLA-DRB1*01:01-KRW reactive CD4+ T cells were detected. This result, in the context of the HLA-F/MHC-I OC pathway, provides experimental data suggesting functional linkage between MHC-I and MHC-II epitopes. The sequences used were:

TST-
[SEQ ID NO: 21]
DIAGTTSTLQEQIGWMTNNPPIPVGEIYKRWIILGLNKIVRMYS
PTSILD
TNA-
[SEQ ID NO: 22]
DIAGTTSNLQEQIAWMTNNPPIPVGEIYKRWIILGLNKIVRMYS
PTSILD
TQT-
[SEQ ID NO: 23]
DIAGTTQTLQEQIGWMTNNPPIPVGEIYKRWIILGLNKIVRMYS
PTSILD

### Example IV

In this Example, interactions between NK receptors and HLA-F were investigated through tetramer and recombinant receptor binding followed by specific receptor/ligand blocking to identify functional interactions. A tumor cell line expressing KIR3DL2 and NK clones expressing combinations of KIR3DL2 and other KIR were used to demonstrate specific functions dependent on the interactions between KIR3DL2 and MHC-I open conformer and HLA-F that resembled but did not precisely mirror the function of inhibitory KIR. These results were extended with NK and T cell lines where functional responses correlated with specific interference of receptor-ligand interaction occurring between KIR3DL2 and both HLA-F and free forms of MHC class I. The data collectively support methods of the present invention involving a broader interaction between MHC-I open conformers—for which HLA-F may serve as the prototypical example—and KIR receptors.

Methods

Cells and Cell lines

B-LCL cell lines were previously collected and analyzed by the International

Histocompatibility Workshops & Conference and obtained directly from the International Histocompatibility Working Group (IHWG) in Seattle (Hansen, 2006, supra). Cell lines were grown in RPMI supplemented with 15% FBS, 100 U/ml penicillin, and 100 U/ml streptomycin. The B-LCL/T cell hybrid cell line T2 (hemizygous chr 6 derived from 0.174, MHC Class II deficient) was obtained from T. Spies (FHCRC, Seattle, Wash.). KIR3DL2+T-cell Actute Lymphoblastic Leukemia line TALL104 was obtained from ATCC and cultured in complete medium (ATCC-formulated IMDM with 20% FCS; 2.5 μg/ml human albumin; 0.5 μg/ml D-mannitol; 100 U rIL2) at a minimum cell density of 5.0×105 cells/ml.

PALL filters of random healthy blood donors were provided by the Puget Sound Blood Center, Seattle Wash., with IRB approval. PBMC were separated from whole blood using density gradient centrifugation with Lymphocyte Separation Medium (Cellgro, Manassas, Va.) and Accuspin columns (Sigma, St Louis, Mo.). NK and T cells were negatively selected using either the NK Isolation Kit II (Miltenyi, Cambridge, Mass.) from enriched PBMC or the Rosettesep™ Human NK cell, CD4 T cell or CD8 T cell Enrichment cocktail (Stemcell Technologies, Vancouver, BC) from whole blood. Purified NK cells were incubated with 100 U rIL-2 for 5-7 days prior to use.

NK cell cloning was performed as described in Cella and Colonna, 2000 Meth. Mol. Biol. 121:1-4 (incorporated herein by reference) after prior enrichment for KIR3DL2 positive NK cells. Negatively enriched NK cells were labeled with mAb Q66 and separated using anti-Mouse IgM microbeads (Miltenyi, Cambridge, Mass.). Enriched KIR3DL2 positive cells were seeded at a concentration of 1 cell/well with 1×10⁵ gamma irradiated PBMC from at least 2 donors and 1×10⁴ gamma irradiated T2 cells as feeders in complete media supplemented with 2 μg/ml PHA and 500 U rIL-2. Cells from wells with an enlarged pellet were expanded, confirmed as NK cells (CD3-CD56+), and screened for expression of KIR.

Functional Assays

Cytotoxicity: B-LCL cell lines were pre-labeled with 50 mCi ⁵¹Cr for 1 hr at 37° C., incubated with blocking mAb or recombinant protein, and plated at 5×103 with effector cells at the indicated effector to target (E:T) ratio. After 16 hr, 30 μl of supernatant was collected and applied to lumaplates, dried, and counted using a TopCount scintillation counter (Perkin Elmer, San Jose Calif.). Background release of chromium was measured from target cells without effectors and full release was taken from targets lysed with 5% triton X-100. Specific lysis was calculated using the formula ((sample-background)/(full release-background))*100.

CD107A and Intracellular IFNg: Assays were performed with purified NK cells at day +7 stimulation with 100 U rIL-2. Target T2 cells were incubated with blocking mAb at saturating concentration as indicated. NK cells were washed and stained with mouse anti-human CD107A-PCy5 (H4A3, BD Pharmingen, San Jose, Calif.) and added to target cells at an E:T ratio of 1:2 for 1 hr at 37° C., and then incubated with monensin (golgistop, BD, San Jose, Calif.) for 5 hr. The cells were then stained and analyzed as described below.

To measure intracellular IFNg, targets and effectors were incubated together for 6 hr at 37° C. and then with 10 μg/ml Brefeldin A for 6 hr at 37° C. Cells were stained for surface phenotype as described below followed by intracellular staining with mouse anti-human IFN-gamma PE-Cy7 (4SB3, BD Pharmingen San Jose, Calif.) using FACS lysing and FACS Permeabilizing solutions (BD, I San Jose, Calif.). CD4 and CD8 positive T cells were incubated with plate bound mouse anti-human CD3 (OKT-3) and 40 U rIL-2 for 7 days prior to assay. IFN-gamma assay was performed as per the NK cell assay minus target cells and incubation of the T cells directly with blocking antibodies.

Statistical testing: Functional assays involving random healthy donors were analyzed using Paired T-test for statistical significance. Data sets showing significant change (P<0.05) in functional response under different conditions are indicated on the graph. All statistical analysis was performed using GraphPad Prism.

Cellular staining and phenotyping: Phenotyping of whole NK cells for functional analysis was performed with direct conjugates of anti-CD56pacific blue (HCD56), anti-KIR2DL1/S1 FITC (HP-MA4), anti-KIR2DL2/3 FITC (DX27), anti-KIR3DL1 A700 (DX9) (Biolegend, San Diego, Calif.) and anti-NKG2A APC (Z199, Beckman Coulter, Brea, Calif.). For CD4 and CD8 T cell assays, mouse anti-human CD4 PE-Cy5 or mouse anti-human CD8 PE-Cy5 were included. Cells were analyzed using a BD LSR-II Flow Cytometer and Flowjo (Tree Star, Inc. Ashland, Oreg.).

For each tetramer staining reaction, 1 μg BirA biotinylated monomer was incubated with 1.5 μg streptavidin-PE (SAPE, Invitrogen, Carlsbad USA) for 30 mM at room temperature. Tetramers were incubated with target cells for 25 mM at 4° C. Tetramer binding was blocked by incubating target cells with blocking mAb or dilution factor (hybridoma supernatant) for 25 min at 4° C. prior to staining with tetramer.

Staining with recombinant KIR was performed by first incubating target cells with blocking mAb or isotype control for 25 mM at 4° C., and then with recombinant KIR for 25 mM at 4° C. KIR binding was detected using streptavidin PE and stained cells analyzed using a FACScan flow cytometer (Becton Dickinson, Mountain View, Calif.) and FlowJo.

Two-step antibody staining was performed with saturating concentrations of biotinylated mAb (typically 10 μg/ml) followed by washing and labeling with streptavidin-PE. mAbs 3D11 specific for HLA-F were generated as previously described (Lee and Geraghty, 2003). HCA2 was from T. Spies (FHCRC). mAb 5.133 (KIR3DL1, KIR3DL2 and KIR2DS4 specific) and mAb DX31 (KIR3DL2 specific) were obtained from K. Campbell and K. Malmberg, respectively. Q66 hybridoma supernatant was provided by D. Pende, Istituto Nazionale per la Ricerca sul Cancro, Genoa, Italy.

Protein Analysis

Refolding and purification: KIR3DL2 cDNA encoding residues 96-319 (D1D2 domains and stem region) was synthesized (Blue Heron, Bothell, Wash.) and cloned with a C-terminus HIS-tag in pET-22b vector. BL21 (DE3) pLysS cells carrying the plasmid were grown to logarithmic phase, induced with 1.0 mM IPTG, and lysed by freezing and thawing. Inclusion bodies were washed extensively to remove contaminating proteins, dissolved in 6 M guanidine hydrocloride, and refolded by dilution into 100 ml refolding buffer (100 mM Tris.HCl pH 8.2, 500 mM L-Arginine HCl, 2 mM EDTA, 6.4 mM cysteamine, 3.6 mM cystamine.2HCl, and 0.1 mM PMSF) to a final concentration of 4 μM and incubated with stirring at 4° C. for 72 h. Refolded protein was dialyzed at 4° C. against 100 mM urea and then against 10 mM Tris.HCl, 10 mM MES, and 100 mM urea before being concentrated using a 0.22 μm filter. KIR3DL2 recombinant protein was purified by ion metal affinity chromatography using Ni-NTA resin (QIAGEN) and then on a Superdex 200 10/300 GL (GE Healthcare) liquid chromatography gel filtration column

Biotinylation: The following were added stepwise to 3.3 ml of KIR3DL2-D1D2stem-bio: 10 mM MgOAc, 10 mM ATP, 50 μM d-biotin, 9 μg BirA, 0.1 mM PMSF, 50 μg leupeptin, and 5 μg pepstalin. After incubation at 21° C. for 17 hr, protein was buffer exchanged into 3.5 ml 20 mM Tris.HCl pH 8.0, 150 mM NaCl, and 2 mM EDTA using a PD10 column and then concentrated to 250 μL.

3DL2 pull-down and Western blotting: Whole cells were incubated with purified KIR3DL2-D1-D2-His for 1 hr at 4° C., lysed in DPBS containing 1% NP40, and the proteins of interest captured by Ni-NTA agarose (Qiagen, Valencia, Calif.). After washing with lysis buffer and then lysis buffer with 10 mM imidazole, protein complexes were eluted with DPBS with 300 mM imidazole. Proteins were separated on 10% Bis-Tris gels (Invitrogen, Grand Island, N.Y.) and analyzed by Western blot using indicated antibodies.

Surface plasmon resonance (SPR) analysis: Interaction between KIR3DL2 D1D2stem and HLA-F/MHCs was analyzed by SPR using a Biacore 3000 system at 25° C. in HBS-EP buffer (10 mM HEPES, 150 mM NaCl, 3 mM EDTA, 0.005% surfactant P20, pH 7.4). SPR ligands—MHCs and KIR3DL2—were biotinylated through an engineered C-terminal BirA biotinylation site. Biotinylated MHCs were purified at the NIH Tetramer Core Facility or the Immune Monitoring Laboratory at FHCRC (Seattle, Wash.). Ligands were capture-immobilized (at 10 μl/min) on a SA sensor chip (˜1000 RU) immediately following repurification by size exclusion chromatography (SEC) to remove any aggregation products. Reference flow cells were left blank. SPR analytes—KIR3DL2D1D2stem, HLA-F, ILT2D1D2, and ILT4D1D2—were repurified by SEC in HBS-EP buffer within 48 h of use.

Experiments were performed in duplicate and run at 20 μl/min After running analytes over the captured MHC surface, a mild acid treatment consisting of two consecutive injections of 10 mM glycine-HCl pH 2.0 (flow rate of 100 μl/min for 5 s) was performed, followed by a 15 mM HBS-EP buffer stabilization and then all analytes were injected again. Analytes and buffer blank injections were randomized before and after acid treatment and between the two sets. Antibodies known to bind to the surfaces were injected at the end of each injection set. Sensorgrams obtained from SPR measurements were analyzed using the double-subtraction method described by Myszka 1999 JMR 12:279-284. DNA encoding the first two extracellular domains of ILT2 and ILT4 were obtained from Dr K. Maenaka (Laboratory Biomol. Sci., Hokkaido University). ILT2D1D2 and ILT4D1D2 were purified as described in Shiroishi et al., 2003 Proc Natl Acad Sci USA 100:8856-8861, incorporated herein by reference.

Results

HLA-F Tetramer Binds Specifically to KIR3DL2 Expressing Cells.

The receptor ligand relationship between MHC-I and KIR, combined with putative structural similarities between HLA-F and MHC-I open conformers, suggested a potential relationship between HLA-F and KIR. The 3-domain KIR was believed to have a receptor role based on the well characterized relationships between 2-domain KIR and MHC-I complex and data implicating interactions between the 3-domain KIR and HLA-B*27 homodimers which may resemble MHC-I open conformers. To determine this, HLA-F tetramer was used to query cell lines that expressed KIR but that did not express MHC-I open conformer (OC), as measured by binding to mAb HCA2 or HC10. It had been previously established that HLA-F tetramer binds cells that express MHC-I OC due to a natural physical interaction between these molecules (Goodridge et al., 2010, supra). Specific staining with HLA-F tetramer was observed with human leukemic T cell line TALL-104, known to express KIR3DL2 and found to be MHC-I OC (HCA2 and HC10) negative. Further, HLA-F tetramer binding to TALL-104 was blocked using anti-3DL2 mAbs (3DL2 specific Q66 or 3DL1/2 specific 5.133). Blocking titrated with decreasing mAb concentration; however, the prior application of HLA-F tetramer did not block respective mAb binding.

NK clones were generated and screened for expression of KIR3DL2 or KIR3LD1. Three appropriate clones were examined, two KIR3DL2 positive as measured by reactivity with both Q66 and 5.133 and one KIR3DL1 positive as measured by reactivity with only 5.133. Clones were tested in the resting state, where they lacked expression of HLA-F or MHC-I OC ligands. Reactivity with HLA-F tetramer was demonstrated for both KIR3DL2 clones while none was detected on the clone expressing KIR3DL1. HLA-F tetramer binding to the KIR3DL2 positive clones was reversed by prior addition of 3DL2 reactive mAb.

Recombinant KIR3DL2 binds to HLA-F and MHC-I open conformer: To confirm HLA-F binding to KIR3DL2, a recombinant form of a truncated KIR3DL2 protein was used in binding assays. Through testing of various recombinant forms, it was found that the KIR3DL2 sequence containing the D1 and D2 domains and stem region, with or without a BirA tail, could be stably refolded while other forms were unable to refold, including those with the D0 domain. The KIR3DL2-D1D2stem-BirA recombinant protein bound to an activated T cell clone expressing both HLA-F and MHC-I OC but not to the same cell in the resting state. Binding of recombinant KIR3DL2 could be blocked with either anti-HLA-F mAb 3D11 or anti-MHC-I HC mAb HCA2, suggesting an interaction with both HLA-F and MHC-I OC either individually or possibly as a heteroduplex. The same recombinant KIR3DL2 was tested for the ability to bind and precipitate ligand using cell lines expressing MHC-I complex and that did or did not express either HLA-F or MHC-I open conformer.

The KIR3DL2-D1D2stem-BirA precipitate from this panel of cells was fractionated and probed for the presence of MHC-I and HLA-F using Western analysis. Detectable levels of both MHC-I and HLA-F were precipitated only from cells that expressed both HLA-F and MHC-I OC, consistent with surface binding of KIR3DL2-D1D2stem and its respective specific mAb blocking. While these experiments confirmed binding between MHC-I OC/HLA-F and KIR3DL2, they do not distinguish between direct binding to each individually or an interaction dependent upon heteroduplex between HLA-F and MHC-I, a possible structure suggested by the physical binding observed between these molecules.

Surface plasmon resonance confirms interactions with MHC-I, HLA-F and KIR3DL2: To further test the interactions between HLA-F and KIR3DL2, SPR measurements were performed using either recombinant KIR3DL2-D1D2stem as analyte over immobilized HLA-F surfaces or refolded HLA-F as analyte over biotinylated KIR3DL2-D1D2stem surface. SPR measurements were performed with multiple concentrations of refolded HLA-F or KIR3DL2-D1D2stem as analytes and varying surface densities of HLA-F. The results of these experiments were consistent with specific binding of HLA-F to KIR3DL2-D1D2stem. Binding decreased with decreasing concentrations of either protein as analyte, and KIR3DL2 binding decreased with lowered concentration of immobilized HLA-F. The findings are also consistent with the ability of HLA-F ligand to bind KIR3DL2 receptor directly without dependency on MHC-I.

To examine MHC-I binding to KIR3DL2, two different methods were used to generate MHC-I OC given the inability to refold stable open conformers. Work had shown that open conformer could be formed from complex refolded with conditional ligand after UV treatment and used as analyte in SPR experiments, suggesting similarly treated MHC-I could be immobilized directly on surfaces. Acid treatment was used as an alternative means of generating open conformers on surfaces so that MHC-I alleles for which allele specific conditional ligands have not been characterized could be included for study. Biotinylated HLA-A*03 and HLA-B*07 proteins refolded with conditional ligand peptides before and after UV treatment were immobilized on different surfaces. KIR3DL2-D1D2stem binding was examined before and after acid treatment. KIR3DL2-D1D2stem bound specifically to both HLA-A*03 and HLA-B*07 after either acid treatment or UV exposure but had reduced or absent binding to complex. In parallel with the observed KIR3DL2 binding, examination of surfaces with control proteins and mAbs that recognized complex (ILT2, ILT4, W6/32, and anti-β2m BB2M) and open conformer (ILT4, HCA2, and HC10) confirmed the structures and showed that acid treatment was quantitatively more effective at producing open conformer.

To examine additional MHC-I alleles, including alleles that had previously been implicated as ligands for KIR3DL2, different surfaces were immobilized with recombinant HLA-A*03, A*11, and A*74, each refolded with an allele specific high affinity peptide. Again, analyte KIR3DL2-D1D2stem bound only HLA-A*03 acid treated surface and not complex, consistent with the results found using conditional ligand peptide. Similar results were obtained with HLA-A*11 surfaces; however, no binding was apparent on HLA-A*74 surfaces either as complex or after acid treatment as open conformer. Control experiments confirmed that complex and open conformer surfaces exhibited essentially similar binding parameters for all three alleles. Allele specific binding to KIR might have been predicted considering KIR3DL2 polymorphism and similar allele specific binding such as the KIR2DL1/2/3 and HLA-C1, C2 receptor-ligand pairs and differential binding of the HLA-Bw4, Bw6 ligands with KIR3DL1.

HLA-F and KIR3DL2 function in the presence or absence of other KIR receptors: To test for functional exchange, the KIR3DL2 positive T cell line TALL-104 was used as effector against B-LCL targets with tetramers and specific mAb to potentially block contact between KIR3DL2 and MHC-I or HLA-F Minimal cytolysis was observed over a wide range of effector to target ratios for B-LCL targets PLH (HLA-A*03, B*47) and HOM2 (HLA-A*03, B*27) and inhibition of lysis was significantly reversed in the presence of target-specific mAbs used to block MHC-I or HLA-F. Furthermore, reversal of inhibition was observed in the presence of HLA-F tetramer and KIR3DL2 reactive mAb used to directly block KIR3DL2 on effectors. This pattern of reactivity was mirrored using two NK clones as effectors, each co-expressing KIR3DL2 combined with either KIR2DL1 or KIR2DL2/3 receptor and target B-LCL expressing either HLA-C1 (BM9, HLA-A2/-B35) or HLA-C2 (KOSE HLA-A2/-B35) as KIR2DL ligands. In the absence of ligand for KIR2DL on the target, lysis by the respective effector was enhanced by blocking MHC-I or HLA-F. Alternatively, when the corresponding KIR2DL receptor was present, lysis was controlled by the interaction between HLA-C ligand and KIR2DL receptor and was reversed only by W6/32, which binds HLA-C1 or C2 complex. The choice of HLA-A*02 positive targets in this instance demonstrated that the effect of KIR3DL2 inhibition by HLA-F can occur independently of HLA-A*03.

HLA-F and MHC-I OC modulate KIR3DL2 reactivity towards target and between effectors: Extending these functional findings, IL-2 stimulated whole NK and T cell populations were used that were selected as KIR3DL2+ single positive subsets to measure cytokine production after exposure to HLA-F positive targets. In this case the target was cell line T2, which expresses surface HLA-F and MHC-I open conformer similar to other B-LCL, but has reduced levels of surface MHC-I complex including HLA-E due to deficiency for TAP and Tapasin. Accordingly, NK cells from random healthy donors were isolated and cultured with IL-2, then exposed to T2 cells with and without addition of MHC-I and HLA-F mAbs tested for the presence of CD107A or intracellular IFNg. Both CD107A and intracellular IFNg were significantly increased when either HLA-F (mAb 3D11) or MHC complex (mAb W6/32) was blocked. Blocking by W6/32 suggests that MHC complex also acts as an inhibitory ligand, but this interpretation is confounded by the fact that W6/32 can bind to different conformational forms of peptide bound and peptide free MHC (Giacomini et al., 1997 Tissue Antigens 50:555-566).

Given that activated lymphocytes express both HLA-F and MHC-I OC, similar experiments were performed with IL-2/CD3 activated CD4+ or CD8+ T cells from random healthy donors to assess whether expression of these ligands during immune activation influences receptor-ligand cross-talk responses between effector cells. Even in the absence of target, significant shifts in IFNγ responses between activated effectors were obtained when blocking HLA-F (mAb 3D11) and MHC (mAb W6/32) while broader variation in responses was seen when blocking MHC-I OC (mAb HCA2). The latter result should be considered within the context that HCA2 reacts with only a subset of MHC-I alleles and the individuals used for these experiments expressed different levels of HCA2 reactive MHC-I.

Functional interaction between KIR and MHC-I has been implicated in a range of immunological roles from pregnancy to transplantation and from autoimmunity to infectious disease. An understanding of the expression and function of HLA-F, which goes hand in hand with the expression of free forms of MHC class I, led to KIR-MHC interactions that may be relevant to the inflammatory response. HLA-F is unique among HLA class I in being surface expressed as an open conformer without peptide and β2m (Lee and Geraghty, 2003 supra; Wainwright et al., 2000 supra)—possibly co-dependent on other MHC-I open conformers for surface expression (Goodridge et al., 2010 supra). Indeed, the co-expression of MHC-I OC and HLA-F on activated cells and their potential ability to physically interact suggests overlapping or interdependent functions. Starting with these considerations, this study was intended to obtain evidence for HLA-F as a ligand for immune receptors and to possibly relate the findings to other MHC-I open conformers. The novel findings include uncovering a potential receptor-ligand relationship between KIR3DL2 and HLA-F and other MHC-I open conformers, potentially leading to a broader and more precise definition of KIR function in the activated immune response.

Although receptor-ligand relationships between MHC-I open conformer have been suggested (Arosa et al., 2007 supra), most of these have been envisioned as acting in cis possibly playing a role in stabilizing the MHC-I OC and directing MHC-I internalization. HLA-F has been proposed for such a role, including both stabilization and internalization, either as a co-receptor for MHC-I ligands acquired extracellularly or through HLA-F-specific internalization signals or both (Goodridge et al., 2010 supra). The trans receptor LILRB2 (ILT4) has been found to be a receptor for peptide free MHC-I, binding both folded and free HC forms, and the activating receptor LILRA1, which displayed a preference for binding to HLA-C HC (Jones et al., 2011 J Immunol. 186:2990-2997).

Early work defined a specific receptor-ligand pairing between KIR3DL2 and HLA-A*03/-A*11, presumably as complex with bound peptide, and not other MHC-I allotypes (Brando et al., 2005 J Leukoc Biol. 78:359-371; Dohring et al., 1996 J Immunol. 156:3098-3101; Pende et al., 1996 J Exp Med. 184:505-518). In addition, studies with HLA-A*03 tetramers and KIR3DL2 transfectants suggested that tetramer binding was dependent on the peptide used to form the tetramer (Hansasuta et al., 2004 Eur. J Immunol. 34:1673-1679). The data presented here as part of the present invention appears to conflict with those reports, in particular that not only HLA-A*03 and HLA-A*11 but also HLA-B*07 bind to KIR3DL2 and all do so in the open conformer form and not as complex. However, it may be important to consider that historically most functional studies used to define KIR specificity have been carried out using B-LCL cell lines as targets, which express an activated phenotype including the expression of HLA-F and MHC class I open conformers, (Dohring 1996 supra; Pende et al., 1996 supra; Storkus et al., 1991 Proc Natl Acad Sci USA 88:5989-5992). In these in vitro experiments, restriction of KIR3DL2 to the allotypes defined might be influenced by the differential ability of allelic MHC to interact with HLA-F. An alternative interpretation of peptide-dependent tetramer binding is that refolding with peptides of varying binding affinities affects the stability of tetramer reagents. Certain peptides may confer stable structures, thus predominantly favoring the complex form of MHC-I, while others confer less stability leading to higher proportion of peptide free MHC-I as tetramers. What is being considered peptide dependent binding may in fact be open conformer binding, reflecting instead the pool of peptide free MHC-I in specific peptide-MHC-I tetramer preparations.

Although the data support an inhibitory function for KIR3DL2, evidence suggests alternative functions are also likely (Sivori et al. Blood 2010 116:1637-1647). Previous studies reported that unlike classical KIR interactions, KIR3DL2 single positive cells are hyporesponsive in individuals with the classical HLA-A*03 and/or -A*11 (Fauriat et al., 2008 J Immunol. 181:6010-6019). This would contradict the idea that KIR3DL2 encounters its putative ligand as a peptide bound complex under normal resting conditions and supports the concept that KIR3DL2 positive cells may instead encounter their respective ligand under inflammatory conditions. Whether the functions of KIR3DL2 and classical KIR overlap at all, even without considering the roles played by HLA-F and other MHC-I OC, may be open to question. The expression of KIR3DL2 increases upon activation from basal levels present on resting cells (Chrul et al., 2006 Mediators Inflamm. 46957), and the majority of functional evidence gathered for KIR3DL2 has been performed with activated effectors such as IL-2 activated NK cell populations or KIR3DL2+NK cell clones (Dohring et al., supra; Pende et al., supra). Further, KIR3DL2 can bind CpG oligodeoxynucleotides (ODNs), which are subsequently co-internalized with KIR3DL2 and shuttled to TLR9 resulting in cytokine release (Marcenaro et al., 2011 Adv Exp Med Biol. 780:45-55). Therefore, the ability of KIR3DL2 to respond to the expression of HLA-F and free forms of class I MHC, combined with its down-regulation by ODN suggests that functional responses mediated by KIR3DL2 may be of greater influence during the inflammatory response than the detection of missing self under resting conditions. Down-regulation by ODN could serve a dual purpose in provoking a TLR based pro-inflammatory signal while also reducing a functional interaction between KIR3DL2 and HLA-F/MHC-OC.

The coincident upregulation of KIR3DL2, HLA-F, and MHC-I OC under inflammatory conditions implies communication may occur between KIR3DL2+NK and T cells with activated HLA-F+ lymphocytes directed through KIR3DL2 and HLA-F/MHC-I OC interactions. Such communication has the potential to affect the magnitude and nature of the inflammatory response, and suggests the possibility that KIR3DL2+ cells become “licensed” or experience an increase in functional capacity upon encounter of HLA-F and peptide free MHC under pro-inflammatory conditions. Licensing of NK raises the issue of why inhibitory receptors are specific for self-MHC-I that do not have a ligand pair in certain individuals, as is often the case with the KIR2DL1/2 and HLA-C1, C2 pairings. Similarly, there are activating receptors that are specific for self-MHC-I that through licensing render those NK unresponsive as in the case of KIR2DS1 and HLA-C1 individuals (Fauriat et al., 2010 Blood 115:1166-1174). Alternative possibilities for ligands have been suggested, including pathogen-encoded or other stress activated signals that might provide high affinity ligands (Fauriat, id.; Long and Raj agopalan, 2002 J Exp Med. 196:1399-1402). The finding of HLA-F and MHC-I OC as ligands expressed on activated cells fits precisely with such a possibility.

An example of KIR/MHC-I HC pairing, and the only one reported outside of the work presented here, is that measured between KIR3DL1/KIR3DL2 and HLA-B*27. HLA-B*27:05 is unusual among MHC-I alleles in being expressed constitutively not only as complex, but also as a dimer without peptide or β2m. KIR3DL1 interacts with HLA-B*27 complex while KIR3DL2 interacts with HLA-B*27 expressed as a homodimer without peptide (Kollnberger et al., 2007 Eur J Immunol. 37:1313-1322). Considering the strong association of HLA-B*27 with ankylosing spondylitis (AS) and related autoimmune conditions and that KIR3DL2, expressed as a homodimer, has the potential to sense heteroduplexes of HLA-F and class I MHC, raises the possibility that KIR3DL2 recognition of HLA-B*27 may represent an aberrant function that does not typically occur under resting conditions, leading to immune dysregulation. This possibility may relate to the expression of KIR3DL2 on Th17 CD4 T cells and their apparent increase in responsiveness in patients with AS (Bowness et al., 2011 J Immunol. 186:2672-2680.).

Other potential roles for HLA-F include modification of or interaction with specific HLA-E and HLA-G receptors. HLA-F, -E, -G and -C are co-expressed in extravillous trophoblasts (EVT) that have invaded the maternal decidua in contact with decidual NK cells (dNK) (Ishitani et al., 2003 J Immunol. 171:1376-1384). The ability of HLA-F to associate with class I MHC and KIR3DL2 raises the possibility that HLA-F may be involved in stabilizing receptor/ligand interactions between EVT and dNK during pregnancy, where dNK responses are essential to the immune regulation of pregnancy (Eastabrook et al., 2008 JOGC 30:467-476). As HLA-F and HLA-E physically interact as open conformers, HLA-F might modify the recognition of HLA-E by CD94/NKG2 heterodimers or alternatively that HLA-E may modify HLA-F interaction with KIR3DL2. The structural similarity between KIR2DS4 and KIR3DL2 in the putative ligand binding domains and functional binding with common allelic MHC-I implies they may have overlapping ligands (Graef et al., 2009 J Exp Med. 206:2557-2572), suggesting the former may also act as a receptor for HLA-F/MHC-I OC.

These findings may be relevant to a reconsideration of MHC-I and KIR genetics, extending knowledge of KIR ligands to include specifically HLA-F as the prototypical MHC-I open conformer alongside all MHC-I OCs. The evidence implicating KIR with disease is primarily derived from genetic association data (Kulkarni et al., 2008 Semin. Immunol. 20:343-352). Thus HLA-F should be considered in studies of KIR in complex disease in at least two respects. First, HLA-F coding sequences are conserved but expression levels of HLA-F vary, presumably based on HLA-F genotype where substantial noncoding variation has been identified (Pyo et al., 2006 Immunogenetics 58:241-251). Secondly, since MHC-I OC and HLA-F are co-expressed and the affinities between HLA-F and different MHC-I alleles vary, the potential exists for HLA-F expression levels to be modified by allelic MHC-I OC and vice versa. Thus, not only HLA-F but also coordinated MHC-I variation may be important considerations in obtaining a precise interpretation of MHC-I/KIR genetic associations.

The embodiments of the invention in which an exclusive property or privilege is claimed are defined as follows:

1. A synthetic polypeptide vaccine for targeting an individual's immune response to an antigen of interest, wherein the polypeptide has an amino acid sequence which comprises a carrier epitope that binds to open conformers of the individual's MHC-I molecules and an effector epitope which elicits an immune response to the antigen of interest, and wherein the carrier epitope is MHC-I HLA type specific for the individual.

2. The synthetic polypeptide vaccine of claim 1, wherein the polypeptide is bounded by one or more caspase cleavage sites.

3. The synthetic polypeptide vaccine of claim 1, wherein the effector epitope modulates an immune response to a tumor, pathogen, or autoantigen associated with an autoimmune disorder.

4. The synthetic polypeptide vaccine of claim 1, wherein the polypeptide stimulates a CD8+ cytotoxic T cell response.

5. The synthetic polypeptide vaccine of claim 1, wherein the polypeptide stimulates a CD4+ T helper cell response.

6. The synthetic polypeptide vaccine of claim 1, further comprising an adjuvant or cytokine.

7. The synthetic polypeptide vaccine of claim 1, wherein the effector epitope is a tumor epitope.

8. The synthetic polypeptide vaccine of claim 7, wherein tumor epitope is derived from a tumor antigen of the tumor of the individual.

9. The synthetic polypeptide vaccine of claim 8, wherein the tumor antigen is identified in a proteome expression profile of the individual.

10. The synthetic polypeptide vaccine of claim 1, wherein the polypeptide comprises more than one effector epitope which elicit immune response to the same antigen.

11. The synthetic polypeptide vaccine of claim 1, wherein the polypeptide comprises more than one effector epitopes which elicit immune responses to different antigens.

12. The synthetic polypeptide vaccine of claim 3, wherein the effector epitope modulates an immune response to a minor histocompatibility autoantigen.

13. The synthetic polypeptide vaccine of claim 3, wherein the carrier epitope binds to the open conformer of the individuals MHC-I molecule in the presence of HLA-F.

14. The synthetic polypeptide vaccine of claim 1, wherein the carrier epitope and the effector epitope are from sequences of two different proteins.

15. The synthetic polypeptide vaccine of claim 14, wherein the carrier epitope and the effector epitope are from sequences of two different proteins from two different individuals of the same species.

16. The synthetic polypeptide vaccine of claim 14, wherein the carrier epitope and the effector epitope sequences are from proteins of different species.

17. The synthetic polypeptide vaccine of claim 1, wherein the carrier epitope and the effector epitope are the same sequence.

18. A method for preparing an immunomodulating polypeptide that targets an individual's immune response to an antigen of interest, comprising:
a) determining the individual's MHC class I HLA type;
b) selecting an amino acid sequence which comprises a carrier epitope that binds to MHC-I open conformers of the individual in the presence of HLA-F and is type specific for the individual;
c) selecting an amino acid sequence which comprises an effector epitope of the antigen of interest to which immunomodulation is desired; and
d) synthesizing the immunomodulating polypeptide which comprises the carrier epitope and the effector epitope.

19. The method of claim 18, wherein the immunomodulating polypeptide further comprises one or more caspase sites.

20. The method of claim 19, wherein the immunomodulating polypeptide further comprises compound HLA sites.

21. The method of claim 19, wherein the immunomodulating polypeptide further comprises an amino acid sequence for a marker or reporter epitope.

22. The method of claim 18, further comprising the step of determining the individual's HLA MHC-II type.

23. The method of claim 22, wherein the immunomodulating polypeptide further comprises an epitope which binds the individual's MHC-II molecule.

24. The method of claim 18, wherein the effector epitope modulates an immune response to a tumor, pathogen, or autoantigen associated with an autoimmune disorder.

25. The method of claim 24, wherein the pathogen is viral.

26. The method of claim 25, wherein the viral is a herpes virus, retrovirus, hepatitis virus, or a flavivirus.

27. The method of claim 26, wherein the virus is human cytomegalovirus, hepatitis B or HIV.

28. The method of claim 24, wherein the tumor antigen is from a solid tumor, a hematologic malignancy, or a melanoma.

29. The method of claim 28, wherein the tumor antigen is from a hematological malignancy selected from the group consisting of acute lymphoblastic leukemia, acute myelogenous leukemia, chronic lymphocytic leukemia, chronic myelogenous leukemia, Hodgkin's lymphoma, non-Hodgkin's lymphoma, and myeloma.

30. The method of claim 24, wherein the tumor antigen is identified by proteome expression profiling of tumor cells obtained from the individual.

31. The method of claim 18, wherein the amino acid sequence of the carrier epitope is altered to increase binding affinity of the polypeptide for the MHC-I open conformer of the individual.

32. The method of claim 18, wherein the amino acid sequence of the carrier epitope is altered to decrease binding affinity of the polypeptide for the MHC-I open conformer of the individual.

33. The method of claim 18, wherein the ability of the carrier epitope to bind the MHC-I open conformer is determined on HLA-F positive cells.

34. The method of claim 33, wherein the HLA-F positive cells are obtained from the individual.

35. The method of claim 34, wherein the immunomodulating polypeptide comprises an epitope directed toward stimulating a CD4+ T helper response for the individual.

36. The method of claim 18, wherein the immunomodulating polypeptide comprises an epitope directed toward stimulating a CD8+ cytotoxic T cell response for the individual.

37. A method for directing the immune response of an individual to a target antigen of interest, comprising:
a) determining the individual's MHC class I HLA type; and
b) contacting cells of the individual that have been upregulated for HLA-F expression upon receiving an activating agent, with a synthetic immunomodulating polypeptide which comprises a carrier epitope that binds to open conformers of the individual's MHC-I molecules and an effector epitope which elicits an immune response to the antigen for which immunomodulation is desired, thereby directing the immune response of the individual to said antigen of interest.

38. The method of claim 37, wherein the cells that upregulate HLA-F expression are lymphocyte, monocytes, and dendritic cells.

39. The method of claim 37, wherein the contacting step is performed ex vivo and further comprising the step of returning the cells to the individual.

40. The method of claim 37, wherein the cells are activated to express HLA-F by treatment with CD40 ligand, adjuvant, human TLR ligand, or TNF-alpha and interferon gamma.

41. The method of claim 37, wherein the contacting step is performed in vivo.

42. The method of claim 41, wherein expression of HLA-F is stimulated in the individual by treating with CD40 ligand, adjuvant, human TLR ligand, or TNF-alpha and interferon gamma.

43. The method of claim 41, wherein the effector epitope stimulates an immune response to a tumor or viral antigen.

44. The method of claim 37, wherein the immunomodulating polypeptide further comprises an epitope which binds the individual's MHC-II molecule.

45. A method for downregulating the immune response of an individual to an antigen, comprising:
a) determining the individual's MHC class I HLA type; and
b) contacting cells of the individual that have been upregulated for HLA-F expression with an inhibitory agent which specifically inhibits the expression of HLA-F on the surface of the individual's activated, thereby downregulating the individual's immune response to the antigen.

46. The method of claim 45, wherein the inhibitory agent which inhibits the expression of HLA-F is a monoclonal antibody or binding fragment thereof that specifically binds to the HLA-F heavy chain.

47. The method of claim 45, wherein the monoclonal antibody or binding fragment thereof specifically binds to an extracellular domain of the HLA-F heavy chain.

48. The method of claim 46, wherein the antibody is a single chain antibody, or a binding fragment thereof.

49. The method of claim 45, wherein the cells upregulated for HLA-F expression are lymphocyte, monocytes, and dendritic cells.

50. The method of claim 45, wherein immune response to be downregulated is an inflammatory response.
